(19) United States
(12) Patent Application Publication
(10) Pub. No.: US 2002/0002137 A1
(43) Pub. Date: Jan. 3, 2002

(54) Combination of growth hormone secretagogues and antidepressants

(76) Inventors: Frank Robert Busch, Gales Ferry, CT (US); Willard McKowan Welch JR., Mystic, CT (US)

Correspondence Address: Gregg C. Benson, Pfizer Inc., Patent Department, MS 4159, Eastern Point Road, Groton, CT, 06340, US

(21) Appl. No.: 09/860,786

(22) Filed: May 18, 2001

Related U.S. Application Data
(60) Provisional application No. 60/207,017, filed on May 25, 2000.

(51) Int. Cl. A61K 38/05; A61K 31/4745

(52) U.S. Cl. 514/19; 514/303

(57) ABSTRACT

This invention is directed to combinations comprising a growth hormone secretagogue, a prodrug thereof or a pharmaceutically acceptable salt of said growth hormone secretagogue or said prodrug and an antidepressant, a prodrug thereof or a pharmaceutically acceptable salt of said antidepressant or said prodrug and to pharmaceutical compositions and kits comprising such combinations. Antidepressants within the scope of this invention include norepinephrine reuptake inhibitors (e.g., secondary and tertiary amine tricyclics), selective sertraline reuptake inhibitors, agents which are combined norepinephrine/sertraline reuptake inhibitors, monoamine oxidase inhibitors and atypical antidepressants. This invention is also directed to methods of improving the physical and/or psychological condition of a patient undergoing a medical procedure, to methods of treating musculoskeletal frailty, to methods of treating congestive heart failure and to methods of attenuating protein catabolic response after a major operation comprising administering such a combination. In particular, this invention relates to such compositions and kits that improve the cardiac function, metabolism, muscle tone and/or mental state of patients undergoing a medical procedure. The compositions and kits of this invention are also useful in treating central nervous system disorders of patients undergoing a medical procedure.

This application claims priority from the provisional application U.S. serial No. 60/207,017, filed on May 25, 2000, the benefit of which is hereby claimed under 37 C.F.R. §1.78(a)(3).

### BACKGROUND OF THE INVENTION

This invention is directed to combinations comprising a growth hormone secretagogue, a prodrug thereof or a pharmaceutically acceptable salt of said growth hormone secretagogue or said prodrug and an antidepressant, a prodrug thereof or a pharmaceutically acceptable salt of said antidepressant or said prodrug and to pharmaceutical compositions and kits comprising such combinations. This inventions is particularly directed to combinations wherein the antidepressant is a selective serotonin reuptake inhibitor. This invention is also directed to methods of improving the physical and/or psychological condition of a patient undergoing a medical procedure, to methods of treating musculoskeletal frailty, to methods of treating congestive heart failure and to methods of attenuating protein catabolic response after a major operation comprising administering such a combination. In particular, this invention relates to such compositions and kits that improve the cardiac function, metabolism, muscle tone and/or mental state of patients undergoing a medical procedure. The compositions and kits of this invention are also useful in treating central nervous system disorders of patients undergoing a medical procedure.

Growth hormone (GH), which is secreted from the pituitary gland, stimulates growth of all tissues of the body that are capable of growing. In addition, GH is known to have the following basic effects on the metabolic process of the body:

1. Increased rate of protein synthesis in substantially all cells of the body;

2. Decreased rate of carbohydrate utilization in cells of the body; and

3. Increased mobilization of free fatty acids and use of fatty acids for energy.

Deficiency in GH results in a variety of medical disorders. In children, it causes dwarfism. In adults, the consequences of acquired GH deficiency include profound reduction in lean body mass and concomitant increase in total body fat, particularly in the truncal region. Decreased skeletal and cardiac muscle mass and muscle strength lead to a significant reduction in exercise capacity. Bone density is also reduced. Administration of exogenous GH has been shown to reverse many of these metabolic changes. Additional benefits of GH therapy have included reduction in LDL cholesterol and improved psychological well-being.

In cases where increased levels of GH were desired, the problem was generally solved by providing exogenous GH or by administering an agent which stimulated GH production and/or release. In either case the peptidyl nature of the compound necessitated that it be administered by injection. Initially the source of GH was the extraction of the pituitary glands of cadavers. This resulted in an expensive product, and carried with it the risk that a disease associated with the source of the pituitary gland could be transmitted to the recipient of the GH (e.g., Jacob-Creutzfeld disease). Recently, recombinant GH has become available which, while no longer carrying any risk of disease transmission, is still a very expensive product which must be given by injection or by a nasal spray.

Most GH deficiencies are caused by defects in GH release, not primary defects in pituitary synthesis of GH. Therefore, an alternative strategy for normalizing serum GH levels is by stimulating its release from somatotrophs. Increasing GH secretion can be achieved by stimulating or inhibiting various neurotransmitter systems in the brain and hypothalamus. As a result, the development of synthetic GH-releasing agents to stimulate pituitary GH secretion are being pursued, and may have several advantages over expensive and inconvenient GH replacement therapy. By acting along physiologic regulatory pathways, the most desirable agents would stimulate pulsatile GH secretion, and excessive levels of GH that have been associated with the undesirable side effects of exogenous GH administration would be avoided by virtue of intact negative feedback loops.

Physiologic and pharmacologic stimulators of GH secretion include arginine, L-3,4-dihydroxyphenylalanine (L-DOPA), glucagon, vasopressin, and insulin induced hypoglycemia, as well as activities such as sleep and exercise, and any activity which indirectly causes GH to be released from the pituitary by acting on the hypothalamus perhaps either to decrease somatostatin secretion or to increase the secretion of the known secretagogue GH releasing factor (GHRF) or an unknown endogenous GH-releasing hormone or all of these.

Tang et al., Restoring and Maintaining Bone in Osteogenic Female Rat Skeleton: I. Changes in Bone Mass and Structure, J. Bone Mineral Research 7 (9), p1093-1104, 1992 discloses data for the lose, restore and maintain (LRM) concept, a practical approach for reversing existing osteoporosis. The LRM concept uses anabolic agents to restore bone mass and architecture (+ phase) and then switches to an agent with the established ability to maintain bone mass, to keep the new bone (+/− phase). The rat study described therein utilized PGE₂ and risedronate, a bisphosphonate, to show that most of the new cancellous and cortical bone induced by PGE₂ can be maintained for at least 60 days after discontinuing PGE₂ by administering risedronate.

Antidepressants are agents used to treat affective or mood disorders and related conditions. Affective mood disorders are characterized by changes in mood as the primary clinical manifestation. Either extreme of mood may be associated with psychosis, manifested as disordered or delusional thinking and perceptions which are often incongruent with the predominant mood. Affective disorders include major depression and mania, including bipolar manic-depressive illness. Preferred classes of antidepressants include norepinephrine reuptake inhibitors (NERIs), including secondary and tertiary amine tricyclics; selective sertraline reuptake inhibitors; combined NERI/SSRIs; monoamine oxidase (MAO) inhibitors; and atypical antidepressants. NERIs potentiate the actions of biogenic amines by blocking their major means of physiological inactivation, which involves transport or reuptake into nerve terminals, and specifically, agents which block the reuptake of norepinephrine into said nerve terminals. The term selective serotonin reuptake inhibitor refers to a compound which inhibits the reuptake of serotonin by afferent neurons. Combined NERI/SSRIs block the reuptake of both serotonin and norepinephrine by afferent neurons. Monoamine oxidase inhibitors are compounds which inhibit monoamine oxidase, for example by blocking the metabolic deamination of a variety of monoamines by mitochondrial monoamine oxidase.

### SUMMARY OF THE INVENTION

This invention is directed to combinations comprising a growth hormone secretagogue (GHS), a prodrug thereof or a pharmaceutically acceptable salt of said GHS or said prodrug and an antidepressant, a prodrug thereof or a pharmaceutically acceptable salt of said antidepressant or said prodrug. This invention is also directed to pharmaceutical compositions, methods and kits comprising said combination, as described below. Preferred classes of antidepressants for use in the combinations, pharmaceutical compositions, kits and methods of this invention are norepinephrine reuptake inhibitors (NERIs), selective serotonin reuptake inhibitors (SSRIs), monoamine oxidase inhibitors (MAO inhibitors), combined NERI/SSRIs, and atypical antidepressants, prodrugs of said antidepressants and pharmaceutically acceptable salts of said antidepressants and said prodrugs.

This invention is particularly directed to pharmaceutical compositions comprising a GHS, a prodrug thereof or a pharmaceutically acceptable salt of said GHS or said prodrug; a SSRI, a prodrug thereof or a pharmaceutically acceptable salt of said SSRI or said prodrug; and a pharmaceutically acceptable carrier, vehicle or diluent.

NERIs are especially preferred. NERIs may be either secondary amine tricyclic compounds or tertiary amine tricyclic compounds. Particularly preferred secondary amine tricyclic NERI compounds include, but are not limited to, amoxapine, desipramine, maprotiline, nortriptyline and protriptyline, prodrugs of said secondary amine tricyclic NERIs and pharmaceutically acceptable salts of said secondary amine tricyclic NERIs and said prodrugs. Particularly preferred tertiary amine tricyclic NERI compounds include, but are not limited to, amitryptiline, clomipramine, doxepin, imipramine and trimipramine, prodrugs of said tertiary amine tricyclic NERIs and pharmaceutically acceptable salts of said tertiary amine tricyclic NERIs and said prodrugs.

SSRIs are also especially preferred. Particularly preferred SSRIs include, but are not limited to, citalopram, femoxetine, fluoxetine, fluvoxamine, indalpine, indeloxazine, milnacipran, paroxetine, sertraline, sibutramine and zimeldine, prodrugs of said SSRIs and pharmaceutically acceptable salts of said SSRIs and said prodrugs. Sertraline and fluoxetine, and pharmaceutically acceptable salts thereof, are more particularly preferred. Sertraline hydrochloride is most preferred.

Combined NERI/SSRIs are also especially preferred. A particularly preferred combined NERI/SSRI is venlafaxine, prodrugs thereof and pharmaceutically acceptable salts of venlafaxine and of said prodrugs. Other combined NERI/SSRIs are also within the scope of the combinations, pharmaceutical compositions, kits and methods of this invention.

Monoamine oxidase (MAO) inhibitors are also especially preferred. Particularly preferred MAO inhibitors include, but are not limited to, phenelzine, tranylcypromine and selegiline, prodrugs thereof and pharmaceutically acceptable salts of said MAO inhibitors and of said prodrugs.

Atypical antidepressants are also especially preferred. Particularly preferred atypical antidepressants include, but are not limited to, bupropion, nefazodone and trazodone, prodrugs thereof and pharmaceutically acceptable salts of said atypical antidepressants and of said prodrugs.

In the combinations, pharmaceutical compositions, methods and kits of this invention, it is preferred that said GHS is a compound of the formula I:

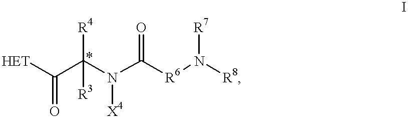

or a stereoisomeric mixture thereof, diastereomerically enriched, diastereomerically pure, enantiomerically enriched or enantiomerically pure isomer thereof,

wherein:

HET is a heterocyclic moiety selected from the group consisting of

[chemical structure]

d is 0, 1 or 2;

e is 1 or 2;

f is 0 or 1;

n and w are 0, 1 or 2, provided that n and w cannot both be 0 at the same time;

Y² is oxygen or sulfur;

A is a divalent radical, where the left hand side of the radical as shown below is connected to C″ and the right hand side of the radical as shown below is connected to C′, selected from the group consisting of

—NR²—C(O)—NR²—, —NR²—S(O)₂—NR²—, —O—C(O)—NR²—, —NR²—C(O)—O—, —C(O)—NR²—C(O)—, —C(O)—NR²—C(R⁹R¹⁰)—, —C(R⁹R¹⁰)—NR²—C(O)—, —C(R⁹R¹⁰)—C(R⁹R¹⁰)—C(R⁹R¹⁰)—, —S(O)₂—C(R⁹R¹⁰)—C(R⁹R¹⁰)—, —C(R⁹R¹⁰)—O—C(O)—, —C(R⁹R¹⁰)—O—C(R⁹R¹⁰)—, —NR²—C(O)—C(R⁹R¹⁰)—, —O—C(O)—C(R⁹R¹⁰)—, —C(R⁹R¹⁰)—C(O)—NR²—, —C(O)—NR²—C(O)—, —C(R⁹R¹⁰)—C(O)—O—, —C(O)—NR²—C(R⁹R¹⁰)—C(R⁹R¹⁰)—, —C(O)—O—C(R⁹R¹⁰)—, —C(R⁹R¹⁰)—C(R⁹R¹⁰)—C(R⁹R¹⁰)—C(R⁹R¹⁰)—, —S(O)₂—NR²—C(R⁹R¹)—C(R⁹R¹⁰)—, —C(R⁹R¹⁰)—C(R⁹R¹⁰)—NR²—C(O)—, —C(R⁹R¹⁰)—C(R⁹R¹⁰)—O—C(O)—, —NR²—C(O)—C(R⁹R¹⁰)—C(R⁹R¹⁰)—, —NR²—S(O)₂—C(R⁹R¹⁰)—C(R⁹R¹⁰)—, —O—C(O)—C(R⁹R¹⁰)—C(R⁹R¹⁰)—, —C(R⁹R¹⁰)—C(R⁹R¹⁰)—C(O)—NR², —C(R⁹R¹⁰)—C(R⁹R¹⁰)—C(O)—, —C(R⁹R¹⁰)—NR²—C(O)—, —C(R⁹R¹⁰)—O—C(O)—NR²—, —C(R⁹R¹⁰)—NR²—C(O)—NR²—, —NR²—C(O)—O—C(R⁹R¹⁰)—, —NR2—C(O)—NR²—C(R⁹R¹⁰)—, —NR²—S(O)₂—NR²—C(R⁹R¹⁰)—, —O—C(O)—NR²—C(R⁹R¹⁰)—, —C(O)—N═C(R¹)—NR²—, —C(O)—NR²—C(R¹1)═N—, —C(R⁹R¹⁰)—NR²—C(R⁹R¹⁰)—, —NR ²C(R⁹R¹⁰)—, —NR²—C(R⁹R¹⁰)—C(R⁹R¹⁰)—, —C(O)—O—C(R⁹R¹⁰)—C(R⁹R¹⁰)—, —NR²—C(R¹1)=N—C(O)—, —C(R⁹R¹⁰)—C(R⁹R¹⁰)—N(R¹²) —C(R⁹R¹⁰)—NR¹²—, —N═C(R¹1)—NR²—C(O)—, —C(R⁹R¹)—C(R⁹R¹⁰)—NR²—S(O)₂—, —C(R⁹R¹⁰)—C(R⁹R¹⁰)—S(O)₂—NR²—, —C(R⁹R¹⁰)—C(R⁹R¹⁰)—C(O)—O—, —C(R⁹R¹⁰)—S(O)₂—C(R⁹R )—, —C(R⁹R¹⁰)—C(R⁹R¹⁰)—S(O)₂—, —O—C(R⁹R¹⁰)—C(R⁹R¹⁰)—, —C(R⁹R¹⁰)—C(R⁹R¹⁰)—O—, —C(R⁹R¹⁰)—C(O)—C(R⁹R¹⁰)—, —C(O)—C(R⁹R)—C(R⁹R¹⁰)— and —C(R⁹R¹⁰)—NR²S(O)₂—NR²;

Q is a covalent bond or CH₂;

W is CH or N;

X is CR⁹R¹⁰, C═CH₂or C═O;

Y is CR⁹R¹⁰, 0 or NR²;

Z is C═O, C═S or S(O)₂;

G¹ is hydrogen, halo, hydroxy, nitro, amino, cyano, phenyl, carboxyl, —CONH₂, —(C₁-C₄)alkyl optionally independently substituted with one or more phenyl, one or more halogens or one or more hydroxy groups, —(C₁-C₄)alkoxy optionally independently substituted with one or more phenyl, one or more halogens or one or more hydroxy groups, —(C₁-C₄)alkylthio, phenoxy, —COO(C₁-C₄)alkyl, N, N-di—(C₁-C₄)alkylamino, —(C₂-C₆)alkenyl optionally independently substituted with one or more phenyl, one or more halogens or one or more hydroxy groups, —(C₂-C₆)alkynyl optionally independently substituted with one or more phenyl, one or more halogens or one or more hydroxy groups, —(C₃-C₆)cycloalkyl optionally independently substituted with one or more (C₁-C₄)alkyl groups, one or more halogens or one or more hydroxy groups, —(C₁-C₄)alkylamino carbonyl or di—(C₁-C₄)alkylamino carbonyl;

G² and G³ are each independently selected from the group consisting of hydrogen, halo, hydroxy, —(C₁-C₄)alkyl optionally independently substituted with one to three halo groups and —(C₁-C₄)alkoxy optionally independently substituted with one to three halo groups;

R¹is hydrogen, —CN, —(CH₂)_{q}N(X⁶)C(O)X⁶, —(CH₂)_{q}N(X⁶)C(O)(CH₂)ₜ-A¹, —(CH₂)_{q}N(X⁶)S(O)₂(CH₂)ₜA¹, (CH₂)_{q}N (X⁶)S(O)₂X⁶, (CH₂)_{q}N(X⁶)C(O)N(X⁶)(CH₂)ₜA¹, —(CH₂)_{q}N(X⁶)C(O)N(X⁶)(X⁶), (CH₂)_{q}C(O)N(X⁶)(X⁶), (CH₂)_{q}C(O)N(X⁶)(CH₂)ₜA¹, —(CH₂)_{q}C(O)OX⁶, (CH₂)_{q}C(O)O(CH₂)ₜ-A¹X, (CH₂)_{q}OX⁶, (CH₂)_{q}OC(O)X⁶, —(CH₂)_{q}OC(O)(CH₂-A¹, —(CH₂)_{q}OC(O)N(X⁶)(CH₂)ₜAl, —(CH₂)_{q}OC(O)N(X⁶)(X⁶), —(CH₂)_{q}C(O)X⁶, (CH₂)_{q}C(O)(CH₂)ₜA¹X, (CH₂)_{q}N(X⁶)C(O)OX⁶, —(CH₂)_{q}N(X⁶)S(O)₂N(X⁶)(X⁶), (CH₂)_{q}S(O)ₘX⁶, (CH₂)_{q}S(O)m(CH₂)ₜA¹, —(C₁-C₁₀)alkyl, —(CH₂)ₜ-A¹, —(CH₂)_{q}—(C₃-C⁷)cycloalkyl, (CH₂)_{q}-Y¹—(C₁-C₆)alkyl, —(CH₂)_{q}—Y¹—(CH₂)ₜ-A¹ or —(CH₂)_{q}—Y¹—(CH₂)ₜ—(C₃-C⁷)cycloalkyl;

where the alkyl and cycloalkyl groups in the definition of R¹ are optionally substituted with (C₁-C₄)alkyl, hydroxy, (C₁-C₄)alkoxy, carboxyl, —CONH₂, —S(O)ₘ(C₁-C₆)alkyl, —CO₂(C₁-C₄)alkyl ester, 1 H-tetrazol-5-yl or 1, 2 or 3 fluoro groups;

Y¹ is O, S(O)ₘ, —C(O)NX⁶—, —CH═CH—, —C≡C—, —N(X⁶)C(O)—, —C(O)NX⁶—, —C(O)O—, —OC(O)N(X⁶)— or —OC(O)—;

q is 0, 1, 2, 3 or 4;

t is 0, 1, 2 or 3;

said (CH₂)_{q} group and (CH₂)ₜ group in the definition of R¹ are optionally independently substituted with hydroxy, (C₁-C₄)alkoxy, carboxyl, —CONH₂, —S(O)ₘ(C₁-C₆)alkyl, —CO₂(C₁-C₄)alkyl ester, 1H-tetrazol-5-yl, 1, 2 or 3 fluoro groups or 1 or 2 (C₁-C₄)alkyl groups;

R^{1A} is selected from the group consisting of hydrogen, F, Cl, Br, I, (C₁-C₆)alkyl, phenyl(C₁-C₃)alkyl, pyridyl(C₁-C₃)alkyl, thiazolyl(C₁-C₃)alkyl and thienyl(C₁-C₃)alkyl, provided that R^{1A} is not F, Cl, Br or I when a heteroatom is vicinal to C″;

R² is hydrogen, (C₁-C₈)alkyl, —(C₀-C₃)alkyl—(C₃-C₈)cycloalkyl, —(C₁-C₄)alkyl-A¹ or A⁻¹;

where the alkyl groups and the cycloalkyl groups in the definition of R² are optionally substituted with hydroxy, —C(O)OX⁶, —C(O)N(X⁶)(X⁶), —N(X⁶)(X⁶), —S(O)ₘ(C₁-C₆)alkyl, —C(O)A¹, —C(O)(X⁶), CF₃, CN or 1, 2 or 3 independently selected halo groups;

R³ is selected from the group consisting of A¹, (C₁-C₁₀)alkyl, —(C₁-C₆)alkyl-A¹, —(C₁-C₆)alkyl-(C₃-C₇)cycloalkyl, —(C₁-C₅)alkyl-X¹-(C₁-C₅)alkyl, —(C₁-C₅)alkyl-X¹-(C₀-C₅)alkyl-A¹ and —(C₁-C₅)alkyl-X¹—(C₁-C₅)alkyl-(C₃-C⁷)cycloalkyl;

where the alkyl groups in the definition of R³ are optionally substituted with —S(O)ₘ(C₁-C₆)alkyl, —C(O)OX³, 1, 2, 3, 4 or 5 independently selected halo groups or 1, 2 or 3 independently selected —OX³ groups;

X¹ is O, S(O)ₘ, —N(X²)C(O)—, —C(O)N(X²)—, —OC(O)—, —C(O)O—, —CX²═CX²—, —N(X²)C(O)O—, —OC(O)N(X²)— or —C≡C—;

R⁴ is hydrogen, (C₁-C₆)alkyl or (C₃-C₇)cycloalkyl, or R⁴ is taken together with R³ and the carbon atom to which they are attached and form (C₅-C₇)cycloalkyl, (C₅-C₇)cycloalkenyl, a partially saturated or fully saturated 4- to 8-membered ring having 1 to 4 heteroatoms independently selected from the group consisting of oxygen, sulfur and nitrogen, or is a bicyclic ring system consisting of a partially saturated or fully saturated 5- or 6-membered ring, fused to a partially saturated, fully unsaturated or fully saturated 5- or 6-membered ring, optionally having 1 to 4 heteroatoms independently selected from the group consisting of nitrogen, sulfur and oxygen;

X⁴ is hydrogen or (C₁-C₆)alkyl or X⁴ is taken together with R⁴ and the nitrogen atom to which X⁴ is attached and the carbon atom to which R⁴ is attached and form a five to seven membered ring;

R⁶ is a bond or is

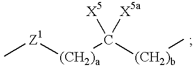

X⁵ and X^{5a} are each independently selected from the group consisting of hydrogen, CF₃, A¹ and optionally substituted (C₁-C₆)alkyl;

the optionally substituted (C₁-C₆)alkyl in the definition of X⁵and X^{5a} is optionally substituted with a substituent selected from the group consisting of A¹, OX², —S(O)ₘ(C₁-C₆)alkyl, —C(O)OX², (C₃-C₇)cycloalkyl, —N(X²)(X²) and —C(O)N(X²)(X²);

or the carbon bearing X⁵ or X^{5a} forms one or two alkylene bridges with the nitrogen atom bearing R⁷ and R⁸ wherein each alkylene bridge contains 1 to 5 carbon atoms, provided that when one alkylene bridge is formed then only one of X⁵ or X^{5a} is on the carbon atom and only one of R⁷ or R⁸ is on the nitrogen atom and further provided that when two alkylene bridges are formed then X⁵ and X^{5a} cannot be on the carbon atom and R⁷ and R⁸ cannot be on the nitrogen atom;

or X⁵ is taken together with X^{5a} and the carbon atom to which they are attached and form a partially saturated or fully saturated 3- to 7-membered ring, or a partially saturated or fully saturated 4- to 8-membered ring having 1 to 4 heteroatoms independently selected from the group consisting of oxygen, sulfur and nitrogen;

or X⁵ is taken together with X^{5a} and the carbon atom to which they are attached and form a bicyclic ring system consisting of a partially saturated or fully saturated 5- or 6-membered ring, optionally having 1 or 2 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;

Z¹ is a bond, O or N—X^{2,} provided that when a and b are both 0 then Z¹ is not N—X² or O; or

R⁶ is —(CR^{a}R^{b})ₐ-E-(CR^{a}R^{b})b, where the —(CR^{a}R^{b})ₐ— group is attached to the carbonyl carbon of the amide group of the compound of formula I and the —(CR^{a}R^{b})_{b} group is attached to the terminal nitrogen atom of the compound of formula I;

E is —O—, —S—, —CH═CH— or an aromatic moiety selected from

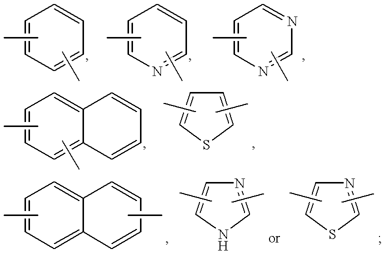

said aromatic moiety in the definition of E optionally substituted with up to three halo, hydroxy, —N(R^{c})(R^{c}), (C₁-C₆)alkyl or (C₁-C₆)alkoxy;

R^{a} and R^{b} are, for each occurrence, independently hydrogen, (C₁-C₆)alkyl, trifluoromethyl, phenyl or monosubstituted (C₁-C₆)alkyl where the substituents are imidazolyl, naphthyl, phenyl, indolyl, p-hydroxyphenyl, —OR^{c}, S(O)ₘR^{c}, C(O)OR^{c}, (C₃-C₇)cycloalkyl, —N(R^{c})(R^{c}), —C(O)N(R^{c})(R^{c}), or R^{a} or R^{b} may independently be joined to one or both of R⁷ or E (where E is other than O, S or —CH═CH—) to form an alkylene bridge between the terminal nitrogen and the alkyl portion of the R^{a} or R^{b} and the R⁷ or E group, wherein the bridge contains 1 to 8 carbon atoms; or R^{a} and R^{b} may be joined to one another to form a (C₃-C₇)cycloalkyl;

R^{c}, for each occurrence, is independently hydrogen or (C₁-C₆)alkyl;

a and b are independently 0, 1, 2 or 3, with the proviso that if E is —O— or —S—, b is other than 0 or 1 and with the further proviso that if E is —CH═CH—, b is other than 0;

R⁷ and R⁸ are each independently hydrogen or optionally substituted (C₁-C₆)alkyl;

where the optionally substituted (C₁-C₆)alkyl in the definition of R⁷ and R⁸ is optionally independently substituted with A¹, —C(O)O—(C₁-C₆)alkyl, —S(O)ₘ(C₁-C₆)alkyl, 1 to 5 halo groups, 1 to 3 hydroxy groups, 1 to 3 —O—C(O)(C₁-C₁₀)alkyl groups or 1 to 3 (C₁-C₆)alkoxy groups; or

R⁷ and R⁸ can be taken together to form —(CH₂)ᵣ-L-(CH₂)ᵣ—;

where L is C(X²)(X²), S(O)ₘ or N(X²);

R⁹ and R¹⁰ are each independently selected from the group consisting of hydrogen, fluoro, hydroxy and (C₁-C₅)alkyl optionally independently substituted with 1-5 halo groups;

R¹¹ is selected from the group consisting of (C₁-C₅)alkyl and phenyl optionally substituted with 1-3 substitutents each independently selected from the group consisting of (C₁-C₅)alkyl, halo and (C₁-C₅)alkoxy;

R¹² is selected from the group consisting of (C₁-C₅)alkylsulfonyl, (C₁-C₅)alkanoyl and (C₁-C₅)alkyl where the alkyl portion is optionally independently substituted by 1-5 halo groups;

A¹ for each occurrence is independently selected from the group consisting of (C₅-C₇)cycloalkenyl, phenyl, a partially saturated, fully saturated or fully unsaturated 4- to 8-membered ring optionally having 1 to 4 heteroatoms independently selected from the group consisting of oxygen, sulfur and nitrogen and a bicyclic ring system consisting of a partially saturated, fully unsaturated or fully saturated 5- or 6-membered ring, optionally having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;

A¹ for each occurrence is independently optionally substituted, on one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N(X⁶)(X⁶), —N(X⁶)C(O)(X⁶), —S(O)₂N (X⁶)(X⁶), —N(X⁶)S(O)₂-phenyl, —N(X⁶)S(O)₂X⁶, —CONX¹¹X¹², —S(O)₂NX¹¹X¹², —NX⁶S(O)₂X¹², —NX⁶CONX¹¹X¹², —NX⁶S(O)₂NX¹¹X¹², —NX⁶C(O)X¹², imidazolyl, thiazolyl and tetrazolyl, provided that if A¹ is optionally substituted with methylenedioxy then it can only be substituted with one methylenedioxy;

where X¹¹ is hydrogen or optionally substituted (C₁-C₆)alkyl;

the optionally substituted (C₁-C₆)alkyl defined for X¹¹ is optionally independently substituted with phenyl, phenoxy, (C₁-C₆)alkoxycarbonyl, —S(O)ₘ(C₁-C₆)alkyl, 1 to 5 halo groups, 1 to 3 hydroxy groups, 1 to 3 (C₁-C₁0)alkanoyloxy groups or 1 to 3 (C₁-C₆)alkoxy groups;

X¹² is hydrogen, (C₁-C₆)alkyl, phenyl, thiazolyl, imidazolyl, furyl or thienyl, provided that when X¹² is not hydrogen, the X¹² group is optionally substituted with one to three substituents independently selected from the group consisting of Cl, F, CH₃, OCH₃, OCF₃ and CF₃;

or X¹¹ and X¹² are taken together to form —(CH₂)ᵣ-L¹-(CH₂)ᵣ—;

L¹ is C(X²)(X²), O, S(O)ₘ or N(X²);

r for each occurrence is independently 1, 2 or 3;

X² for each occurrence is independently hydrogen, optionally substituted (C₁-C₆)alkyl or optionally substituted (C₃-C₇)cycloalkyl, where the optionally substituted (C₁-C₆)alkyl and optionally substituted (C₃-C₇)cycloalkyl in the definition of X² are optionally independently substituted with —S(O)ₘ(C₁-C₆)alkyl, —C(O)OX³, 1 to 5 halo groups or 1-3 OX³ groups;

X³ for each occurrence is independently hydrogen or (C₁-C₆)alkyl;

X⁶ for each occurrence is independently hydrogen, optionally substituted (C₁-C₆)alkyl, (C₂-C₆)halogenated alkyl, optionally substituted (C₃-C₇)cycloalkyl, (C₃-C₇)halogenated cycloalkyl, where optionally substituted (C₁-C₆)alkyl and optionally substituted (C₃-C₇)cycloalkyl in the definition of X⁶ is optionally independently mono- or di-substituted with (C₁-C₄)alkyl, hydroxy, (C₁-C₄)alkoxy, carboxyl, CONH₂, —S(O)ₘ(C₁-C₆)alkyl, carboxylate (C₁-C₄)alkyl ester or 1 H-tetrazol-5-yl; or when there are two X⁶ groups on one atom and both X⁶ are independently (C₁-C₆)alkyl, the two (C₁-C₆)alkyl groups may be optionally joined and, together with the atom to which the two X⁶ groups are attached, form a 4- to 9-membered ring optionally having oxygen, sulfur or NX⁷ as a ring member;

X⁷ is hydrogen or (C₁-C₆)alkyl optionally substituted with hydroxy;

m for each occurrence is independently 0, 1 or 2;

with the proviso that:

X⁶ and X¹² cannot be hydrogen when attached to C(O) or S(O)₂in the form C(O)X⁶, C(O)X¹², S(O)₂X⁶ or S(O)₂X¹²; and

when R⁶ is a bond then L is N(X²) and each r in the definition —(CH₂)ᵣ-L-(CH₂)ᵣ— is independently 2 or 3;

a prodrug thereof or a pharmaceutically acceptable salt of said compound or of said prodrug.

In the combinations, pharmaceutical compositions, methods and kits of this invention, it is especially preferred that said GHS is a compound of the formula

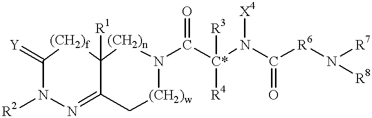

or a stereoisomeric mixture thereof, diastereomerically enriched, diastereomerically pure, enantiomerically enriched or enantiomerically pure isomer thereof,

wherein:

wherein

f is 0;

n is 0 and w is 2, or n is 1 and w is 1, or n is 2 and w is 0;

Y is oxygen or sulfur;

R¹ is hydrogen, —CN, —(CH₂)_{q}N(X⁶)C(O)X⁶, (CH₂)_{q}N(X⁶)C(O)(CH₂)ₜ-A¹, —(CH₂)_{q}N(X⁶)SO₂(CH₂)ₜ-A¹, (CH₂)_{q}N(X⁶)S0₂X⁶, —(CH₂)_{q}N(X⁶)C(O)N(X⁶)(CH₂)ₜ-A¹, —(CH₂)_{q}N(X⁶)C(O)N(X⁶)(X⁶), (CH₂)_{q}C(O)N(X⁶)(X⁶), (CH₂)_{q}C(O)N(X⁶)(CH₂)ₜ-A¹, (CH₂)_{q}C(O)OX⁶—(CH₂)_{q}C(O)O(CH₂)ₜ-A¹, (CH₂)_{q}OX⁶, —(CH₂)_{q}OC(O)X⁶, —(CH₂)_{q}OC(O)(CH₂)ₜ-A¹, —(CH₂)_{q}OC(O)N(X⁶)(CH₂)ₜ-A¹, —(CH₂)_{q}OC(O)N(X⁶)(X⁶), (CH₂)_{q}C(O)X⁶, (CH₂)_{q}C(O)(CH₂)ₜ-A¹, (CH₂)_{q}N(X⁶)C(O)OX⁶, —(CH₂)_{q}N(X⁶)SO₂N(X⁶)(X⁶), (CH₂)_{q}S(O)ₘX⁶—(CH₂)_{q}S(O)ₘ(CH₂)ₜ-A¹, —(C₁-C₁₀)alkyl, —(CH₂)ₜ-A, —(CH₂)_{q}—(C₃-C₇)cycloalkyl, —(CH₂)_{q}—Y—(C₁-C₆)alkyl, (CH₂)_{q}-Y¹l—(CH₂)ₜ-A¹ or —(CH₂)_{q}—Y¹—(CH₂)ₜ—(C₃-C₇)cycloalkyl;

where the alkyl and cycloalkyl groups in the definition of R¹ are optionally substituted with (C₁-C₄)alkyl, hydroxyl, (C₁-C₄)alkoxy, carboxyl, —CONH₂, —S(O)ₘ(C₁-C₆)alkyl, —CO₂(C₁-C₄)alkyl ester, 1H-tetrazol-5-yl or 1, 2 or 3 fluoro;

Y¹ is O, S(O)ₘ, —C(O)NX⁶—, —CH═CH—, —C≡C—, —N(X⁶)C(O)—, —C(O)NX⁶—, —C(O)O—, —OC(O)N(X⁶)— or —OC(O)—;

q is 0, 1, 2, 3 or 4;

t is 0, 1, 2 or 3;

said (CH₂)_{q} group and (CH₂)ₜ group may each be optionally substituted with hydroxyl, (C₁-C₄)alkoxy, carboxyl, —CONH₂, —S(O)ₘ(C₁-C₆)alkyl, —CO₂(C₁-C₄)alkyl ester, 1H-tetrazol-5-yl, 1, 2 or 3 fluoro, or 1 or 2 (C₁-C₄)alkyl;

R² is hydrogen, (C₁-C₈)alkyl, —(C₀-C₃)alkyl-(C₃-C₈)cycloalkyl, —(C₁-C₄)alkyl-A¹ or A¹;

where the alkyl groups and the cycloalkyl groups in the definition of R² are optionally substituted with hydroxyl, —C(O)OX⁶, —C(O)N(X⁶)(X⁶), —N(X⁶)(X⁶), —S(O)ₘ(C₁-C₆)alkyl, —C(O)A, —C(O)(X⁶), CF₃, CN or 1, 2 or 3 halogen;

R³ is A¹, (C₁-C₁₀)alkyl, —(C₁-C₆)alkyl-A, —(C₁-C₆)alkyl-(C₃-C₇)cycloalkyl, —(C₁-C₅)alkyl-X—(C₁-C₅)alkyl, —(C₁-C₅)alkyl-X—(C₀-C₅)alkyl-A¹ or —(C₁-C₅)alkyl-X—(C₁-C₅)alkyl-(C₃-C₇)cycloalkyl;

where the alkyl groups in the definition of R³ are optionally substituted with, —S(O)ₘ(C₁-C₆)alkyl, —C(O)OX³, 1, 2, 3, 4 or 5 halogens, or 1, 2 or 3 OX³;

X¹is O, S(O)ₘ, —N(X²)C(O)—, —C(O)N(X²)—, —OC(O)—, —C(O)O—, —CX²═CX²—, —N(X²)C(O)O—, —OC(O)N(X²)— or —C≡C—;

R⁴is hydrogen, (C₁-C₆)alkyl or (C₃-C₇)cycloalkyl;

X⁴ is hydrogen or (C₁-C₆)alkyl or X⁴ is taken together with R⁴ and the nitrogen atom to which X⁴ is attached and the carbon atom to which R⁴ is attached and form a five to seven membered ring;

R⁶ is a bond or is

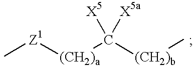

where a and b are independently 0, 1, 2 or 3;

X⁵ and X^{5a} are each independently selected from the group consisting of hydrogen, trifluoromethyl, A¹ and optionally substituted (C₁-C₆)alkyl;

the optionally substituted (C₁-C₆)alkyl in the definition of X⁵ and X^{5a} is optionally substituted with a substituent selected from the group consisting of A¹, OX², —S(O)ₘ(C₁-C₆)alkyl, —C(O)OX², (C₃-C₇)cycloalkyl, —N(X²)(X²) and —C(O)N(X²)(X²);

R⁷ and R⁸ are independently hydrogen or optionally substituted (C₁-C₆)alkyl;

where the optionally substituted (C₁-C₆)alkyl in the definition of R⁷ and R⁸ is optionally independently substituted with A¹, —C(O)O—(C₁-C₆)alkyl, —S(O)ₘ(C₁-C₆)alkyl, 1 to 5 halogens, 1 to 3 hydroxy, 1 to 3 —O—C(O)(C₁-C₁₀)alkyl or 1 to 3 (C₁-C₆)alkoxy; or

R⁷ and R⁸ can be taken together to form —(CH₂)ᵣ-L-(CH₂)ᵣ—;

where L is C(X²)(X²), S(O)ₘ or N(X²);

A¹ in the definition of R¹ is a partially saturated, fully saturated or fully unsaturated 4- to 8-membered ring optionally having 1 to 4 heteroatoms independently selected from the group consisting of oxygen, sulfur and nitrogen, a bicyclic ring system consisting of a partially saturated, fully unsaturated or fully saturated 5- or 6-membered ring, having 1 to 4 heteroatoms independently selected from the group consisting of nitrogen, sulfur and oxygen, fused to a partially saturated, fully saturated or fully unsaturated 5- or 6-membered ring, optionally having I to 4 heteroatoms independently selected from the group consisting of nitrogen, sulfur and oxygen;

A¹ in the definition of R², R³, R⁶, R⁷ and R⁸ is independently (C₅-C₇)cycloalkenyl, phenyl or a partially saturated, fully saturated or fully unsaturated 4- to 8-membered ring optionally having 1 to 4 heteroatoms independently selected from the group consisting of oxygen, sulfur and nitrogen, a bicyclic ring system consisting of a partially saturated, fully unsaturated or fully saturated 5- or 6-membered ring, optionally having 1 to 4 heteroatoms independently selected from the group consisting of nitorgen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;

A¹ for each occurrence is independently optionally substituted, in one or optionally both rings if A¹ is a bicyclic ring system, with up to three substituents, each substituent independently selected from the group consisting of F, Cl, Br, I, OCF₃, OCF₂H, CF₃, CH₃, OCH₃, -OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N(X⁶)(X⁶), —N (X⁶)C(O)(X⁶), —SO₂N(X⁶)(X⁶), —N(X⁶)SO₂-phenyl, —N(X⁶)SO₂X⁶, —CONX IX^{12,} —SO₂NX¹IX¹², —NX⁶S0₂X¹², —NX⁶CONX¹¹X¹², —NX⁶SO₂NX¹¹X¹², —NX⁶C(O)X¹², imidazolyl, thiazolyl or tetrazolyl, provided that if A¹ is optionally substituted with methylenedioxy then it can only be substituted with one methylenedioxy;

where X¹¹ is hydrogen or optionally substituted (C₁-C₆)alkyl;

the optionally substituted (C₁-C₆)alkyl defined for X¹¹ is optionally independently substituted with phenyl, phenoxy, (C₁-C₆)alkoxycarbonyl, —S(O)ₘ(C₁-C₆)alkyl 1 to 5 halogens, 1 to 3 hydroxy, 1 to 3 (C₁-C₁₀)alkanoyloxy or 1 to 3 (C₁-C₆)alkoxy;

X¹² is hydrogen, (C₁-C₆)alkyl, phenyl, thiazolyl, imidazolyl, furyl or thienyl, provided that when X¹² is not hydrogen, X¹² is optionally substituted with one to three substituents independently selected from the group consisting of Cl, F, CH₃, OCH₃, OCF₃ and CF₃;

or X¹¹ and X¹² are taken together to form —(CH₂)ᵣ-L-(CH₂)ᵣ—;

where L¹ is C(X²)(X²), O, S(O)ₘ or N(X²);

r for each occurrence is independently 1, 2 or 3;

X² for each occurrence is independently hydrogen, optionally substituted (C₁-C₆)alkyl, or optionally substituted (C₃-C₇)cycloalkyl, where the optionally substituted (C₁-C₆)alkyl and optionally substituted (C₃-C₇)cycloalkyl in the definition of X² are optionally independently substituted with —S(O)ₘ(C₁-C₆)alkyl, —C(O)OX³, 1 to 5 halogens or 1-3 OX³;

X³ for each occurrence is independently hydrogen or (C₁-C₆)alkyl;

X⁶is independently hydrogen, optionally substituted (C₁-C₆)alkyl, (C₂-C₆)halogenated alkyl, optionally substituted (C₃-C₇)cycloalkyl, (C₃-C₇)-halogenatedcycloalkyl, where optionally substituted (C₁-C₆)alkyl and optionally substituted (C₃-C₇)cycloalkyl in the definition of X⁶ is optionally independently substituted by 1 or 2 (C₁-C₄)alkyl, hydroxyl, (C₁-C₄)alkoxy, carboxyl, CONH₂, —S(O)ₘ(C₁-C₆)alkyl, carboxylate (C₁-C₄)alkyl ester, or 1H-tetrazol-5-yl; or when there are two X⁶ groups on one atom and both X⁶ are independently (C₁-C₆)alkyl, the two (C₁-C₆)alkyl groups may be optionally joined and, together with the atom to which the two X⁶ groups are attached, form a 4- to 9-membered ring optionally having oxygen, sulfur or NX⁷;

X⁷ is hydrogen or (C₁-C₆)alkyl optionally substituted with hydroxyl; and

m for each occurrence is independently 0, 1 or 2;

with the proviso that:

X⁶ and X¹² cannot be hydrogen when it is attached to C(O) or SO₂ in the form C(O)X⁶, C(O)X¹², SO₂X⁶ or SO₂X¹²; and

when R⁶ is a bond then L is N(X²) and each r in the definition —(CH₂)ᵣ-L-(CH₂)ᵣ— is independently 2 or 3.

In the combinations, pharmaceutical compositions, methods and kits of this invention, it is even more especially preferred that said GHS is 2-amino-N-(1 (R)-benzyloxymethyl-2-(1,3-dioxo-8a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide; 2-amino-N-[2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide; or 2-amino-N-(1-(R)-(2,4-difluoro-benzyloxymethyl)-2-oxo-2-(3-oxo-3a-(R)-pyridin-2-ylmethyl)-2-(2,2,2-trifluoroethyl)-2,3,3a,4,6,7-hexahydro-pyrazolo[4,3-c]pyridin-5-yl)-ethyl)-2-methylpropionamide, a prodrug thereof or a pharmaceutically acceptable salt thereof or of said prodrug.

In the combinations, pharmaceutical compositions, methods and kits of this invention, it is still more especially preferred that the L-tartrate salt of 2-amino-N-(1 (R)-benzyloxymethyl-2-(1,3-dioxo-8a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide; the L-tartrate salt of 2-amino-N-(2-(3a(R)-benzyl-2-methyl-3-oxo-2,3, 3a, 4,6,7-hexahydro-pyrazolo-[4,3-c]pyridin-5-yl)-1 (R)-benzyloxymethyl-2-oxo-ethyl)-isobutyramide; or the L-tartrate salt of 2-amino-N-(1-(R)-(2,4-difluoro-benzyloxymethyl)-2-oxo-2-(3-oxo-3a-(R)-pyridin-2-yl)-2-(2,2,2-trifluoro-ethyl)-2,3,3a,4,6,7-hexahydro-pyrazolo-[4,3-c]pyridin-5-yl)-ethyl)-2-methyl-propionamide is used.

In the combinations, pharmaceutical compositions, methods and kits of this invention, it is also preferred that said GHS is hexarelin, ipamorelin, MK-0677, NN703, L-162752, L-163022, GPA-748, KP102, GHRP-2 or LY444711.

This invention is also directed to a method of improving the physical or psychological condition of a patient undergoing a medical procedure comprising administering to said patient:

a) a pharmaceutical composition comprising a GHS, a prodrug thereof or a pharmaceutically acceptable salt of said GHS or of said prodrug, an antidepressant, a prodrug thereof or a pharmaceutically acceptable salt of said antidepressant or of said prodrug, and a pharmaceutically acceptable vehicle, carrier or diluent; or

b) a GHS, prodrug thereof, pharmaceutically acceptable salt of said GHS or of said prodrug or a pharmaceutical composition thereof and an antidepressant, prodrug thereof, pharmaceutically acceptable salt of said antidepressant or said prodrug or a pharmaceutical composition thereof. This invention thus includes methods whereby a fixed combination is administered and methods whereby the individual components of the combination are administered separately. This invention is particularly directed to such methods wherein the cardiac function, metabolism, muscle tone or mental state of said patient is improved.

It is preferred that said medical procedure is a surgical or dental procedure, though patients undergoing other medical procedures which adversely affect the mental state of said patient may also be treated by the methods of this invention. The combination may be administered before, during or after said surgical or dental procedure.

This invention is also directed to a method for treating musculoskeletal frailty in a mammal comprising administering to said mammal:

a) a pharmaceutical composition comprising a GHS, a prodrug thereof or a pharmaceutically acceptable salt of said GHS or of said prodrug, an antidepresant, a prodrug thereof or a pharmaceutically acceptable salt of said antidepressant or of said prodrug, and a pharmaceutically acceptable vehicle, carrier or diluent; or

b) a GHS, prodrug thereof, pharmaceutically acceptable salt of said GHS or of said prodrug or a pharmaceutical composition thereof and an antidepressant, prodrug thereof, pharmaceutically acceptable salt of said antidepressant or said prodrug or a pharmaceutical composition thereof. This invention thus includes methods whereby a fixed combination is administered and methods whereby the individual components of the combination are administered separately. This invention is particularly directed to such methods wherein bone healing following facial reconstruction, maxillary reconstruction or mandibular reconstruction is treated, vertebral synostosis is induced or long bone extension is enhanced, the healing rate of a bone graft is enhanced or prosthetic ingrowth is enhanced. This invention is also particularly directed to such methods wherein muscle mass is increased.

This invention is also directed to a kit comprising:

a) a first unit dosage form comprising a GHS, a prodrug thereof or a pharmaceutically acceptable salt of said GHS or said prodrug and a pharmaceutically acceptable carrier, vehicle or diluent;

b) a second unit dosage form comprising an antidepressant, a prodrug thereof or a pharmaceutically acceptable salt of said antidepressant or said prodrug and a pharmaceutically acceptable carrier, vehicle or diluent; and

c) a container.

This invention is also directed to a method of treating congestive heart failure in a mammal comprising administering to said mammal:

a) a pharmaceutical composition comprising a GHS, a prodrug thereof or a pharmaceutically acceptable salt of said GHS or of said prodrug, an antidepressant, a prodrug thereof or a pharmaceutically acceptable salt of said antidepressant or of said prodrug, and a pharmaceutically acceptable vehicle, carrier or diluent; or

b) a GHS, prodrug thereof, pharmaceutically acceptable salt of said GHS or of said prodrug or a pharmaceutical composition thereof and an antidepressant, prodrug thereof, pharmaceutically acceptable salt of said antidepressant or said prodrug or a pharmaceutical composition thereof. This invention thus includes methods whereby a fixed combination is administered and methods whereby the individual components of the combination are administered separately.

This invention is also directed to a method of attenuating protein catabolic response after a major operation in a mammal comprising adminstering to said mammal:

a) a pharmaceutical composition comprising a GHS, a prodrug thereof or a pharmaceutically acceptable salt of said GHS or of said prodrug, an antidepressant, a prodrug thereof or a pharmaceutically acceptable salt of said antidepressant or of said prodrug, and a pharmaceutically acceptable vehicle, carrier or diluent; or

b) a GHS, prodrug thereof, pharmaceutically acceptable salt of said GHS or of said prodrug or a pharmaceutical composition thereof and an antidepressant, prodrug thereof, pharmaceutically acceptable salt of said antidepressant or said prodrug or a pharmaceutical composition thereof. This invention thus includes methods whereby a fixed combination is administered and methods whereby the individual components of the combination are administered separately.

The phrase “condition which presents with low bone mass” refers to a condition where the level of bone mass is below the age specific normal as defined in standards by the World Health Organization “Assessment of Fracture Risk and its Application to Screening for Postmenopausal Osteoporosis (1994), Report of a World Health Organization Study Group. World Health Organization Technical Series 843′. Childhood idiopathic and primary osteoporosis are also included. Included in the treatment of osteoporosis is the prevention or attenuation of long term complications such as curvature of the spine, loss of height, prosthetic surgery, and prevention of prostate malfunctioning. Also included is increasing the bone fracture healing rate and enhancing the rate of successful bone grafts. Also included is periodontal disease and alveolar bone loss.

The prospect of surgery, whether invasive or non-invasive, often leads to depressed mental states in patients. Such mental states can be detrimental to rapid recovery from the surgical procedure. Patients with depressed mental states or at risk of acquiring a depressed mental state can be treated with the combination of this invention.

The phrase “musculoskeletal frailty” refers to a condition wherein a subject has low bone mass and/or low muscle mass, and includes such diseases, disorders and conditions as, but not limited to, conditions which present with low bone mass, osteoporosis, conditions which present with low muscle mass, osteotomy, childhood idiopathic bone loss, bone loss associated with periodontitis, bone healing following facial reconstruction, maxillary reconstruction, mandibular reconstruction and bone fracture. Further, musculoskeletal frailty encompasses such conditions as interfaces between newly attached prostheses and bone which require bone ingrowth.

The term “pharmaceutically acceptable” means that a substance or mixture of substances must be compatible with the other ingredients of a formulation and not deleterious to a patient.

The term “treating”, “treat” or “treatment” as used herein includes curative, preventative (e.g., prophylactic) and palliative treatment.

The terms “patient” and “subject” are used interchangeably and refer to animals, particularly mammals such as dogs, cats, cattle, horses, sheep and humans. Particularly preferred patients and subjects are humans, including males and females.

The parenthetical negative or positive sign used herein in the nomenclature denotes the direction plane polarized light is rotated by the particular stereoisomer.

The subject invention also includes combinations, pharmaceutical compositions, methods and kits comprising isotopically-labeled compounds, which are identical to the compounds described hereinabove, but for the fact that one or more atoms are replaced by an atom having an atomic mass or mass number different from the atomic mass or mass number usually found in nature. Examples of isotopes that can be incorporated into compounds used in the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸ F and ³⁶Cl, respectively. Compounds used in the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which racioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds used in this invention and prodrugs thereof can generally be prepared by carrying out the procedures disclosed in the Schemes and /or in the Examples and Preparations described in the patents and applications which are incorporated herein by reference, by substituting a readily available isotopically labeled reagent for a non-isotopically labeled reagent.

The combinations, pharmaceutical compositions, kits and methods of this invention increase bone density and muscle mass while at the same time reducing fat mass and total serum cholesterol. Further, the combinations, pharmaceutical compositions, kits and methods of this invention result in improved cardiac output, improved wound healing, higher metabolism and improved mental state which provides for positive outcomes following medical procedures, including surgical and dental procedures. This invention also makes a significant contribution to the art by providing compositions and methods that increase and maintain bone mass resulting in prevention, retardation, and/or regression of osteoporosis and related bone disorders.

Other features and advantages will be apparent from the description and claims which describe the invention.

### DETAILED DESCRIPTION OF THE INVENTION

The first compound of this invention is a growth hormone secretagogue (GHS). Any GHS may be used in the combinations, pharmaceutical compositions, methods and kits of this invention.

A representative first class of growth hormone secretagogues within those compounds of Formula I as described hereinabove is set forth in PCT Application Publication No. WO97/24369, which is incorporated herein by reference, as compounds having the structural formula:

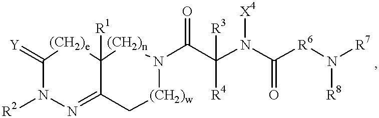

wherein the various substituents are as defined in WO97/24369. Said compounds are prepared as disclosed therein.

2-Amino—N-(2-(3a-(R)-benzyl-2-methyl-3-oxo-2,3,3a ,4,6,7-hexahydro-pyrazolo-[4,3-c]pyridin-5-yl)-1-(R)-benzyloxymethyl-2-oxo-ethyl)-isobutyramide, having the following structure:

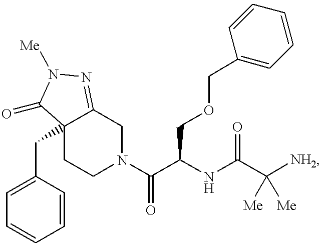

and 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo-[4,3-c]pyridin-5-yl)-ethyl)-2-methyl-propionamide, having the following structure:

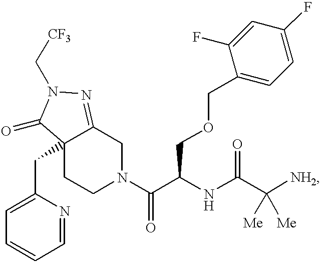

are both within the scope of the disclosure of International Pat. Application Publication No. WO97/24369.

Those compounds of Formula I which are not within the disclosure of International Pat. Application Publication No. WO97/24369 may be prepared as disclosed in International Pat. Application Publication No. WO98/58947, which is incorporated herein by reference.

2-amino-N-(1 (R)-benzyloxymethyl-2-(1 ,3-dioxo-8a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide, having the following structure:

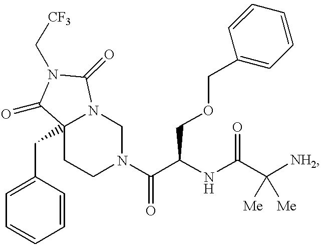

is within the scope of the disclosure of International Pat. Application Publication No. WO98/58947.

Other GHS compounds which may be used in the compositions, methods and kits of this invention include the following:

(1) compounds of the formula

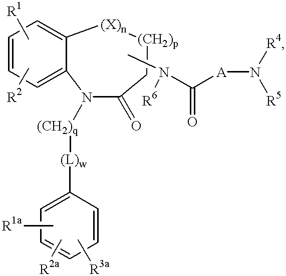

wherein the various substituents are defined, and the compounds are prepared, as disclosed in U.S. Pat. No. 5,206,235, which is incorporated herein by reference;

(2) compounds of the formula

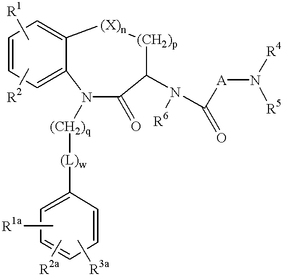

wherein the various substituents are defined, and the compounds are prepared, as disclosed in U.S. Pat. No. 5,283,241, which is incorporated herein by reference;

(3) compounds of the formula

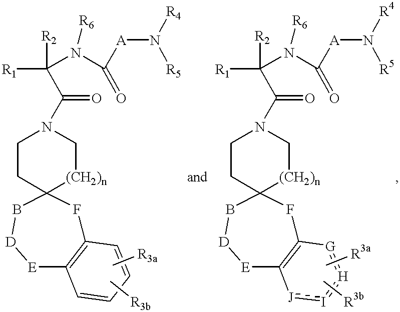

wherein the various substituents are defined, and the compounds are prepared, as disclosed in International Pat. Application Publication No. WO97/41879, which is incorporated herein by reference; and

(4) compounds of the formula

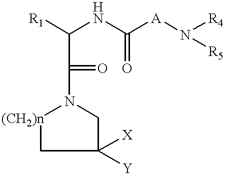

wherein the various substituents are defined, and the compounds are prepared, as disclosed in U.S. Pat. No. 5,492,916, which is incorporated herein by reference.

The most preferred compounds within (1) above have the following structures:

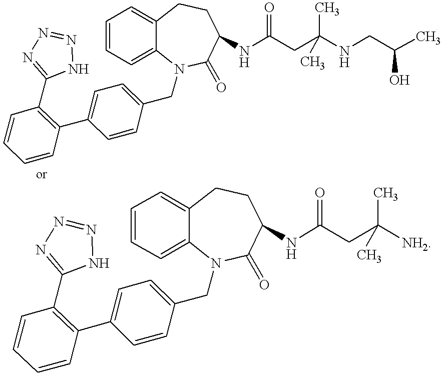

The most preferred compound within (3) above has the following structure:

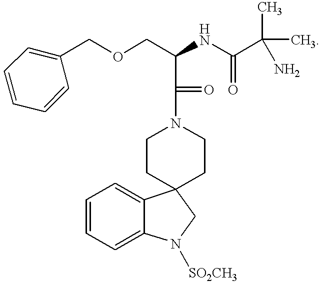

The methanesulfonate salt of this compound is particularly preferred.

Still other compounds which may be used within the compositions, methods and kits of this invention include:

(5) GHRP-6, which is the prototype GH-releasing peptide H-His-D-Trp-Ala-Trp-D-Phe-Lys-NH₂, (also called His¹, Lys⁶)-GHRP), is sold commercially by Bachem, catalog number H-9990 and Peninsula Labs, catalog number 8071 and is disclosed in U.S. Pat. No. 4,411,890, which is incorporated herein by reference, and in Bowers et al., Endocrinology, 114:1537, 1984;

(6) GHRP-1, also known as KP101, which is the second generation GH-releasing peptide Ala-His-D-βNaI-Ala-Trp-D-Phe-Lys-NH₂ and is disclosed in Akman, Endocrinology, 132:1286, 1993;

(7) GHRP-2, also known as KP-102 (Kaken) and GPA-748 (Wyeth-Ayerst), which is the GH-releasing peptide D-Ala-D-βNaI-Ala-Trp-D-Phe-Lys-NH₂ and is disclosed in Bowers et al., Endocrinology, 114:1537, 1984 and in Bowers in: Molecular and Clinical Advances in Pituitary Disorders, pp. 153-157, 1993, edited by S. Melmed, Endocrine Research and Education, Inc., Los Angeles, Calif., USA; and

(8) hexarelin, which is His-D-2-methyl-Trp-Ala-Trp-D-Phe-Lys-NH₂, is sold commercially by Peninsula Labs, catalog number 8083, was synthesized by Europeptides, Argenteuil, France and is disclosed in Guillaume et al., Endocrinology, 135, 1073, 1994.

Any antidepressant may be used in the combinations, pharmaceutical compositions, methods and kits of this invention. The term antidepressant means an agent used to treat affective or mood disorders and related conditions. Affective mood disorders are characterized by changes in mood as the primary clinical manifestation. Either extreme of mood may be associated with psychosis, manifested as disordered or delusional thinking and perceptions which are often incongruent with the predominant mood. Affective disorders include major depression and mania, including bipolar manic-depressive illness. Preferred classes of antidepressants include norepinephrine reuptake inhibitors (NERIs), including secondary and tertiary amine tricyclics; selective sertraline reuptake inhibitors; combined NERI/SSRIs; monoamine oxidase (MAO) inhibitors; and atypical antidepressants.

Any norepinephrine reuptake inhibitor (NERI) may be used in the combinations, pharmaceutical compositions, methods and kits of this invention. The term norepinephrine reuptake inhibitor means agents which potentiate the actions of biogenic amines by blocking their major means of physiological inactivation, which involves transport or reuptake into nerve terminals, and specifically, agents which block the reuptake of norepinephrine into said nerve terminals.

Preferred tertiary amine tricyclic norepinephrine reuptake inhibitors which may be used in accordance with this invention include, but are not limited to, amitriptyline, which may be prepared as described in U.S. Pat. No. 3,205,264; chlomipramine, which may be prepared as described in U.S. Pat. No. 3,467,650; doxepin, which may be prepared as described in U.S. Pat. No. 3,420,851; imipramine, which may be prepared as described in U.S. Pat. No. 2,554,736; and trimipramine, which may be prepared as described in Jacob and Messer, Compt. Rend. 252, 2117 (1961).

Preferred secondary amine tricyclic norepinephrine reuptake inhibitors which may be used in accordance with this invention include, but are not limited to, amoxapine, which may be prepared as described in U.S. Pat. No. 3,663,696; desipramine, which may be prepared as described in U.S. Pat. No. 3,454,554; maprotiline, which may be prepared as described in U.S. Pat. No. 3,999,201; nortriptyline, which may be prepared as described in U.S. Pat. No. 3,442,949; and protriptyline, which may be prepared as described in U.S. Pat. No. 3,244,748.

Any selective serotonin reuptake inhibitor (SSRI) may be used in the combinations, pharmaceutical compositions, methods and kits of this invention. The term selective serotonin reuptake inhibitor refers to a compound which inhibits the reuptake of serotonin by afferent neurons. Such inhibition is readily determined by those skilled in the art according to standard assays such as those disclosed in U.S. Pat. No. 4,536,518 and other U.S. patents recited in the next paragraph.

Preferred selective serotonin reuptake inhibitors (SSRI) which may be used in accordance with this invention include, but are not limited to: citalopram, which may be prepared as described in U.S. Pat. No. 4,136,193; femoxetine, which may be prepared as described in U.S. Pat. No. 3,912,743; fluoxetine, which may be prepared as described in U.S. Pat. No. 4,314,081; fluvoxamine, which may be prepared as described in U.S. Pat. No. 4,085,225; indalpine, which may be prepared as described in U.S. Pat. No. 4,064,255; indeloxazine, which may be prepared as described in U.S. Pat. No. 4,109,088; milnacipran, which may be prepared as described in U.S. Pat. No. 4,478,836; paroxetine, which may be prepared as described in U.S. Pat. No. 3,912,743 or U.S. Pat. No. 4,007,196; sertraline and the hydrochloride salt of sertraline, which may be prepared as described in U.S. Pat. No. 4,536,518; sibutramine, which may be prepared as described in U.S. Pat. No. 4,929,629; and zimeldine, which may be prepared as described in U.S. Pat. No. 3,928,369. Fluoxetine is also known as Prozac®. Sertraline hydrochloride is also known as Zoloft®. Sibutramine is also known as Meridia®.

Any combined NERI/SSRI may be used in the combinations, pharmaceutical compositions, methods and kits of this invention. The term combined NERI/SSRI refers to a compound which blocks the reuptake of both serotonin and norepinephrine by afferent neurons. A preferred combined NERI/SSRI which may be used in accordance with this invention is venlafaxine, which may be prepared as described in U.S. Pat. No. 4,535,186.

Any monoamine oxidase (MAO) inhibitor may be used in the combinations, pharmaceutical compositions, methods and kits of this invention. The term monoamine oxidase inhibitor refers to a compound which inhibits monoamine oxidase, for example by blocking the metabolic deamination of a variety of monoamines by mitochondrial monoamine oxidase. Preferred monoamine oxidase inhibitors which may be used in accordance with this invention include, but are not limited to, phenelzine, which may be prepared as described in U.S. Pat. No. 3,000,903; tranylcypromine, which may be prepared as described in U.S. Pat. No. 2,997,422; and selegiline, which may be prepared as described in U.S. Pat. No. 4,564,706.

Any atypical antidepressant may be used in the combinations, pharmaceutical compositions, methods and kits of this invention. The term atypical antidepressant refers to any antidepressant not within any of the aforesaid classes of antidepressants. Preferred atypical antidepressants which may be used in accordance with this invention include, but are not limited to, bupropion, which may be prepared as described in U.S. Pat. No. 3,885,046; nefazodone, which may be prepared as described in U.S. Pat. No. 4,338,317; and trazodone, which may be prepared as described in U.S. Pat. No. 3,381,009.

The disclosures of each of the patents and published patent applications cited within this description are incorporated herein by reference.

The expression “pharmaceutically acceptable salts” includes both pharmaceutically acceptable acid addition salts and pharmaceutically acceptable cationic salts, where appropriate. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, d-tartrate, I-tartrate, citrate, methanesulfonate (mesylate) and p-toluenesulfonate (tosylate) salts.

Pharmaceutically acceptable cationic salts of the compounds used in this invention may be readily prepared, where appropriate, by reacting the free acid form of said compound with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (sodium or potassium ethylhexanoate, magnesium oleate), and employing a solvent (e.g., ethyl acetate) from which the desired cationic salt precipitates, or can be otherwise isolated by concentration and/or addition of a non-solvent.

The acid addition salts of the compounds used in this invention may be readily prepared by reacting the free base form of said compound with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

In addition, the growth hormone secretagogues and antidepressants which may be used in accordance with this invention, prodrugs thereof and pharmaceutically acceptable salts thereof or of said prodrugs, may occur as hydrates or solvates. Said hydrates and solvates are also within the scope of the invention.

The utility of the combinations, pharmaceutical compositions, kits and methods of the present invention as medical agents in the treatment of musculoskeletal frailty (e.g., conditions which present with low bone mass or low muscle mass including osteoporosis) in mammals (e.g. humans) is demonstrated by the activity of the compounds of this invention in conventional assays as set forth in U.S. Pat. No. 5,552,412 and International Pat. Application Publication No. WO97/24369. Such assays also provide a means whereby the activities of the compositions of this invention can be compared between themselves and with the activities of other known compounds and/or compositions. The results of these comparisons are useful for determining dosage levels in mammals, including humans, for the treatment of such diseases.

Administration of the compounds used in this invention can be via any method which delivers the compounds or the combination of this invention systemically and/or locally. These methods include oral, parenteral, intraduodenal routes, etc. Generally, the compounds used in this invention are administered orally, but parenteral administration (e.g., intravenous, intramuscular, transcutaneous, subcutaneous or intramedullary) may be utilized, for example, where oral administration is inappropriate for the instant target or where the patient is unable to ingest the drug. The two different compounds used in this invention can be co-administered simultaneously or sequentially in any order, or a single pharmaceutical composition comprising a first compound as described above and a second compound as described above in a pharmaceutically acceptable carrier can be administered.

In any event the amount and timing of compounds administered will, of course, be dependent on the subject being treated, on the severity of the affliction, on the manner of administration and on the judgment of the prescribing physician. Thus, because of patient to patient variability, the dosages given below are a guideline and the physician may titrate doses of the drug to achieve the activity (e.g., muscle mass improvement, mental state improvement and/or metabolism improvement) that the physician considers appropriate for the individual patient. In considering the degree of activity desired, the physician must balance a variety of factors such as muscle mass starting level, cardiac output, age of the patient, presence of preexisting disease, other ongoing or planned medical treatments or procedures, as well as the presence of other diseases. The following paragraphs provide preferred dosage ranges for the various components of this invention.

This invention relates both to methods of treating the physical and mental condition of a patient and/or to improve the cardiac function, metabolism and muscle condition of a patient in which the GHS and antidepressant are administered together, as part of the same pharmaceutical composition, and to methods in which these two agents are administered separately, as part of an appropriate dosage regimen designed to obtain the benefits of the combination therapy. The appropriate dosage regimen, the amount of each dose administered and the intervals between doses of the active agents will depend upon the GHS and the antidepressant being used, the type of pharmaceutical formulations being used, the characteristics of the subject being treated and the severity of the complications. Generally, in carrying out the methods of this invention, an effective dosage for the GHS compounds of this invention is in the range of 0.0002 to 2 mg/kg/day, preferably 0.01 to 1 mg/kg/day in single or divided doses. It is preferred that the dosage amount of said GHS is about 1 mg to about 50 mg per day for an average subject, depending upon the GHS and the route of administration. The GHS compound and the antidepressant will be administered in single or divided doses. The preferred dosage ranges for the antidepressants used in this invention will vary depending upon the particular antidepressant used. The preferred dosage amounts of the antidepressants are well known to those skilled in the art or can be found in the Physicians Desk References (PDR®), 54^{th} Edition, 2000, Medical Economics Company, Inc., Montvale, N.J., 07645 or in Goodman and Gilman's The Pharmacological Basis of Therapeutics, Hardman, Limbird, Molinoff, Ruddon and Gilman, Eds., 9^{th} Edition, 1996, McGraw-Hill, New York, pp. 433-435. For example, SSRIs will generally be administered in amounts ranging from about 0.05 mg/kg/day to about 10 mg/kg/day in single or divided doses, preferably 5 mg to about 500 mg per day for an average subject, depending upon the SSRI and the route of administration. However, some variation in dosage will necessarily occur depending on the condition of the subject being treated. The prescribing physician will, in any event, determine the appropriate dose for the individual subject.

Pharmaceutical compositions comprising a growth hormone secretagogue, a prodrug thereof or a pharmaceutically acceptable salt of said growth hormone secretagogue or said prodrug and an antidepressant, a prodrug thereof or a pharmaceutically acceptable salt of said antidepressant or said prodrug are hereinafter referred to, collectively, as “the active compositions of this invention.”

Where the tartrate salt, hydrochloride salt or other pharmaceutically acceptable salt of any of the above compounds is used in this invention, the skilled person will be able to calculate effective dosage amounts by calculating the molecular weight of the salt form and performing simple stoichiometric ratios.

The compounds, prodrugs and pharmaceutically acceptable salts used in the combinations of the present invention are generally administered in the form of a pharmaceutical composition comprising at least one of the compounds or pharmaceutically acceptable salts thereof of this invention together with a pharmaceutically acceptable vehicle or diluent. Thus, the compounds, prodrugs and pharmaceutically acceptable salts thereof of this invention can be administered separately or together in any conventional oral, parenteral or transdermal dosage form. When administered separately, the administration of the other compound or a pharmaceutically acceptable salt thereof of the invention follows.

For oral administration a compound or pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often useful for tableting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds or pharmaceutically aceptable salts thereof of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of each active ingredient are known, or will be apparent in light of this disclosure, to those skilled in this art. For examples, see Remington's Pharmaceutical Sciences, Mack Publishing Company, Easton, Pa., 19th Edition (1995).

Pharmaceutical compositions according to the invention may contain 0.1%-95% of a combination of the compounds, prodrugs or pharmaceutically acceptable salts thereof used in this invention, preferably 1%-70%. In any event, the composition or formulation to be administered will contain a quantity of a combination of the compounds, prodrugs or pharmaceutically acceptable salts thereof used in the invention in an amount effective to treat the disease/condition of the subject being treated.

Since the present invention relates to treatment with a combination of the two active ingredients which may be administered separately, the invention also relates to combining separate pharmaceutical compositions in kit form. The kit includes two separate pharmaceutical compositions: a GHS, a prodrug thereof or a pharmaceutically acceptable salt thereof or of said prodrug and an antidepressant, a prodrug thereof or a pharmaceutically acceptable salt thereof or of said prodrug. The kit includes a container for containing the separate compositions such as a divided bottle or a divided foil packet, however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It is desirable to provide a memory aid on a card insert, e.g., in the form of numbers next to the tablets or capsules whereby the numbers correspond with the days of the regimen which the tablets or capsules so specified should be ingested. Another example of such a memory aid is a calendar printed on the card e.g., as follows “First Week, Monday, Tuesday, . . . etc . . . Second Week, Monday, Tuesday, . . . ” etc. Other variations of memory aids will be readily apparent. A “daily dose” can be a single tablet or capsule or several pills or capsules to be taken on a given day. Also a daily dose of antidepressant can consist of one tablet or capsule while a daily dose of a GHS can consist of several tablets or capsules and vice versa. The memory aid should reflect this.

In another specific embodiment of the invention a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

It should be understood that the invention is not limited to the particular embodiments described herein, but that various changes and modifications may be made without departing from the spirit and scope of this invention as defined by the following claims.

1. A combination comprising a growth hormone secretagogue (GHS), a prodrug thereof or a pharmaceutically acceptable salt of said GHS or said prodrug and an antidepressant, a prodrug thereof or a pharmaceutically acceptable salt of said antidepressant or said prodrug.

2. A combination of claim 1 wherein said antidepressant is a norepinephrine reuptake inhibitor (NERI), selective serotonin reuptake inhibitor (SSRI), monoamine oxidase inhibitor (MAO), combined NERI/SSRI, or an atypical antidepressant, a prodrug of said antidepressant or a pharmaceutically acceptable salt of said antidepressant or said prodrug.

3. A combination of claim 2 wherein said antidepressant is a selective serotonin reuptake inhibitor (SSRI), a prodrug thereof or a pharmaceutically acceptable salt of said SSRI or said prodrug.

4. A combination of claim 3 wherein said SSRI is citalopram, femoxetine, fluoxetine, fluvoxamine, indalpine, indeloxazine, milnacipran, paroxetine, sertraline, sibutramine or zimeldine, a prodrug of said SSRI or a pharmaceutically acceptable salt of said SSRI or said prodrug.

5. A combination of claim 4 wherein said SSRI is sertraline, a prodrug thereof or a pharmaceutically acceptable salt of sertraline or of said prodrug.

6. A combination of claim 1 wherein said GHS is a compound of the formula I: 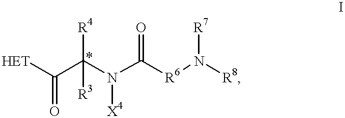
or a stereoisomeric mixture thereof, diastereomerically enriched, diastereomerically pure, enantiomerically enriched or enantiomerically pure isomer thereof,
wherein:
HET is a heterocyclic moiety selected from the group consisting of [chemical structure]
d is 0, 1 or 2;
e is 1 or 2;
f is 0 or 1;
n and w are 0, 1 or 2, provided that n and w cannot both be 0 at the same time;
Y² is oxygen or sulfur;
A is a divalent radical, where the left hand side of the radical as shown below is connected to C″ and the right hand side of the radical as shown below is connected to C′, selected from the group consisting of
—NR²—C(O)—NR²—, —NR²—S(O)₂—NR²—, —O—C(O)—NR²—, —NR²—C(O)—O—, —C(O)—NR²—C(O)—, —C(O)—NR²—C(R⁹R¹⁰)—, —C(R⁹R¹⁰)—NR²—C(O)—, —C(R⁹R¹⁰)—C(R⁹R¹⁰)—C(R⁹R¹⁰)—, —S(O)₂—C(R⁹R¹⁰)—C(R⁹R¹⁰)—, —C(R⁹R¹⁰)—O—C(O)—, —C(R⁹R¹⁰)—O—C(R⁹R¹⁰)—, —NR²—C(O)—C(R⁹R¹⁰)—, —O—C(O)—C(R⁹R¹⁰)—, —C(R⁹R¹⁰)—C(O)—NR²—, —C(O)—NR²—C(O)—, —C(R⁹R¹⁰)—C(O)—O—, —C(O)—NR²—C(R⁹R¹⁰)—C(R⁹R¹⁰)—, —C(O)—O—C(R⁹R¹⁰)—, —C(R⁹R¹⁰)—C(R⁹R¹⁰)—C(R⁹R¹⁰)—C(R⁹R¹⁰)—, —S(O)₂—NR²—C(R⁹R¹⁰)—C(R⁹R¹⁰)—, —C(R⁹R¹⁰)—C(R⁹R¹⁰)—NR²—C(O)— —C(R⁹R¹⁰)—C(R⁹R¹⁰)—O—C(O)—, —NR²—C(O)—C(R⁹R¹⁰)—C(R⁹R¹⁰)—, —NR²—S(O)₂—C(R⁹R¹⁰)—C(R⁹R¹⁰)—, —O—C(O)—C(R⁹R¹⁰)—C(R⁹R¹⁰)—, —C(R⁹R¹⁰)—C(R⁹R¹⁰)—C(O)—NR²—, —C(R⁹R¹⁰))—C(R⁹R¹⁰)—C(O)—, —C(R⁹R¹⁰)—NR²—C(O)—O—, —C(R⁹R¹⁰)—O—C(O)—NR², —C(R⁹R¹⁰)—NR²—C(O)—NR²—, —NR²—C(O)—O—C(R⁹R¹⁰)—, —N R²—C(O)—N R²—C(R⁹R¹⁰)—, —NR²—S(O)₂—NR²—C(R⁹R)—, -Q—C(O)—NR —C(R⁹R¹⁰)—, —C(O)—N═C(R¹)—NR²—, —C(O)—NR²—C(R¹¹)═N—, —C(R⁹R¹⁰)—NR^{12—C(R}⁹R¹⁰)—, —NR¹²C(R⁹R¹⁰)—, —NR^{12C(R}⁹R¹⁰)—C(R⁹R¹⁰)—, —C(O)—O—C(R⁹R¹⁰)—C(R⁹R)—, —NR —C(R )═N—C(Q)—, —C(R⁹R¹⁰)—C(R⁹R¹⁰)—N(R^{12)—, —C(R}⁹R¹⁰)—NR^{12-, —N═C(R}¹⁰)—NR²—C(O)—, —C(R⁹R¹⁰)—C(R⁹R¹⁰)—N R²—S(O)₂—, —C(R⁹R¹⁰)—C(R⁹R¹⁰)—S(O)₂—N R²—, —C(R⁹R¹⁰)—C(R⁹R¹⁰)—C(O)—O—, —C(R⁹R¹⁰)—S(O)₂—C(R⁹R¹⁰)—, —C(R⁹R¹⁰)—C(R⁹R¹⁰)—S(O)₂—, —O—C(R⁹R¹⁰)—C(R⁹R¹⁰)—, —C(R⁹R¹⁰)—C(R⁹R¹⁰)—O—, —C(R⁹R¹⁰)—C(O)—C(R⁹R¹⁰)—, —C(O)—C(R⁹R¹⁰)—C(R⁹R¹⁰)— and —C(R⁹R¹⁰)—NR²—S(O)₂—NR²—;
Q is a covalent bond or CH₂;
W is CH or N;
X is CR⁹R¹⁰, C═CH₂ or C═O;
Y is CR⁹R¹⁰, O or NR²;
Z is C═O, C═S or S(O)₂;
G¹ is hydrogen, halo, hydroxy, nitro, amino, cyano, phenyl, carboxyl, —CONH₂, —(C₁-C₄)alkyl optionally independently substituted with one or more phenyl, one or more halogens or one or more hydroxy groups, —(C₁-C₄)alkoxy optionally independently substituted with one or more phenyl, one or more halogens or one or more hydroxy groups, —(C₁-C₄)alkylthio, phenoxy, —COO(C₁-C₄)alkyl, N,N-di—(C₁-C₄)alkylamino, —(C₂-C₆)alkenyl optionally independently substituted with one or more phenyl, one or more halogens or one or more hydroxy groups, —(C₂-C₆)alkynyl optionally independently substituted with one or more phenyl, one or more halogens or one or more hydroxy groups, —(C₃-C₆)cycloalkyl optionally independently substituted with one or more (C₁-C₄)alkyl groups, one or more halogens or one or more hydroxy groups, —(C₁-C₄)alkylamino carbonyl or di-(C₁-C₄)alkylamino carbonyl;
G²and G³ are each independently selected from the group consisting of hydrogen, halo, hydroxy, —(C₁-C₄)alkyl optionally independently substituted with one to three halo groups and —(C₁-C₄)alkoxy optionally independently substituted with one to three halo groups;
R¹ is hydrogen, —CN, (CH₂)_{q}N(X⁶)C(O)X⁶, (CH₂)_{q}N(X⁶)C(O)(CH₂)ₜ-A¹, (CH₂)_{q}N(X⁶)S(O)₂(CH₂)ₜ-A¹, (CH₂)_{q}N(X⁶)S(O)₂X⁶, —(CH₂)_{q}N(X⁶)C(O)N(X⁶)(CH₂)ₜ-A¹, (CH₂)_{q}N(X⁶)C(O)N(X⁶)(X⁶), (CH₂)_{q}C(O)N(X⁶)(X⁶), (CH₂)_{q}C(O)N(X⁶)(CH₂)ₜ-A¹, (CH₂)_{q}C(O)OX⁶, (CH₂)_{q}C(O)O(CH₂)ₜ-A¹l, (CH₂)_{q}OX⁶, —(CH₂)_{q}OC(O)X⁶, (CH₂)_{q}OC(O)(CH₂)ₜ-A¹, (CH₂)_{q}OC(O)N(X⁶)(CH₂)ₜ-A¹, (CH₂)_{q}OC(O)N(X⁶)(X⁶), (CH₂)_{q}C(O)X⁶, (CH₂)_{q}C(O)(CH₂)ₜ-A¹7, (CH₂)_{q}N(X⁶)C(O)OX⁶, —(CH₂)_{q}N(X⁶)S(O)₂N(X⁶)(X⁶), (CH₂)_{q}S(O)ₘ,X⁶, 7(CH₂)_{q}S(O)ₘ(CH₂)ₜ-A¹, —(C₁-C₁₀)alkyl, —(CH₂)ₜ-A¹, —(CH₂)_{q}—(C₃-C₇)cycloalkyl, —(CH₂)_{q}—Y¹—(C₁-C₆)alkyl, (CH₂)_{q}Y¹—(CH₂)ₜ-A¹or —(CH₂)_{q}—Y¹—(CH₂)t—(C₃-C₇)cycloalkyl;
where the alkyl and cycloalkyl groups in the definition of R¹ are optionally substituted with (C₁-C₄)alkyl, hydroxy, (C₁-C₄)alkoxy, carboxyl, —CONH₂, —S(O)ₘ(C₁-C₆)alkyl, —CO₂(C₁-C₄)alkyl ester, 1H-tetrazol-5-yl or 1, 2 or 3 fluoro groups;
Y¹is O, S(O)ₘ, —C(O)NX⁶—, —CH≡CH—, —C≡C—, —N(X⁶)C(O)—, —C(O)NX⁶—, —C(O)O—, —OC(O)N(X⁶)— or —OC(O)—;
q is 0, 1, 2, 3 or 4;
t is 0, 1, 2 or 3;
said (CH₂)_{q} group and (CH₂)ₜ group in the definition of R are optionally independently substituted with hydroxy, (C₁-C₄)alkoxy, carboxyl, —CONH₂, —S(O)ₘ(C₁-C₆)alkyl, —CO₂(C₁-C₄)alkyl ester, 1H-tetrazol-5-yl, 1, 2 or 3 fluoro groups or 1 or 2 (C₁-C₄)alkyl groups;
R^{1A} is selected from the group consisting of hydrogen, F, Cl, Br, I, (C₁-C₆)alkyl, phenyl(C₁-C₃)alkyl, pyridyl(C₁-C₃)alkyl, thiazolyl(C₁-C₃)alkyl and thienyl(C₁-C₃)alkyl, provided that R^{1A} is not F, Cl, Br or I when a heteroatom is vicinal to C″; R² is hydrogen, (C₁-C₈)alkyl, —(C₀-C₃)alkyl-(C₃-C₈)cycloalkyl, —(C₁-C₄)alkyl-A¹or A¹;
where the alkyl groups and the cycloalkyl groups in the definition of R²are optionally substituted with hydroxy, —C(O)OX⁶, —C(O)N(X⁶)(X⁶), —N(X⁶)(X⁶), —S(O)ₘ(C₁-C₆)alkyl, —C(O)A¹, —C(O)(X⁶), CF₃, CN or 1, 2 or 3 independently selected halo groups;
R³ is selected from the group consisting of A¹, (C₁-C₁₀)alkyl, —(C₁-C₆)alkyl-A¹, —(C₁-C₆)alkyl-(C₃-C₇)cycloalkyl, —(C₁-C₅)alkyl-X¹—(C₁-C₅)alkyl, —(C₁-C₅)alkyl-X¹—(C₀-C₅)alkyl-A¹ and —(C₁-C₅)alkyl-X¹—(C₁-C₅)alkyl-(C₃-C₇)cycloalkyl;
where the alkyl groups in the definition of R³ are optionally substituted with —S(O)ₘ(C₁-C₆)alkyl, —C(O)OX³, 1, 2, 3, 4 or 5 independently selected halo groups or 1, 2 or 3 independently selected —OX³ groups;
X¹ is O, S(O)ᵣ, —N(X²)C(O)—, —C(O)N(X²)—, OC(O)—, —C(O)O—, —CX²═CX²—, —N (X²)C(O)O—, —OC(O)N(X²)— or —C≡C—;
R⁴ is hydrogen, (C₁-C₆)alkyl or (C₃-C₇)cycloalkyl, or R⁴is taken together with R³ and the carbon atom to which they are attached and form (C₅-C₇)cycloalkyl, (C₅-C₇)cycloalkenyl, a partially saturated or fully saturated 4- to 8-membered ring having 1 to 4 heteroatoms independently selected from the group consisting of oxygen, sulfur and nitrogen, or is a bicyclic ring system consisting of a partially saturated or fully saturated 5- or 6-membered ring, fused to a partially saturated, fully unsaturated or fully saturated 5- or 6-membered ring, optionally having 1 to 4 heteroatoms independently selected from the group consisting of nitrogen, sulfur and oxygen; X⁴is hydrogen or (C₁-C₆)alkyl or X⁴is taken together with R⁴and the nitrogen atom to which X⁴is attached and the carbon atom to which R⁴is attached and form a five to seven membered ring;
R⁶ is a bond or is 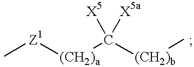
X⁵and X^{5a} are each independently selected from the group consisting of hydrogen, CF₃, A and optionally substituted (C₁-C₆)alkyl;
the optionally substituted (C₁-C₆)alkyl in the definition of X⁵and X^{5a} is optionally substituted with a substituent selected from the group consisting of A₁, OX², —S(O)ₘ(C₁-C₆)alkyl, —C(O)OX², (C₃-C₇)cycloalkyl, —N(X²)(X²) and —C(O)N(X²)(X²);
or the carbon bearing X⁵or X^{5a} forms one or two alkylene bridges with the nitrogen atom bearing R⁷ and R⁸ wherein each alkylene bridge contains 1 to 5 carbon atoms, provided that when one alkylene bridge is formed then only one of X⁵ or X^{5a} is on the carbon atom and only one of R⁷ or R⁸ is on the nitrogen atom and further provided that when two alkylene bridges are formed then X⁵ and X^{5a} cannot be on the carbon atom and R⁷ and R⁸ cannot be on the nitrogen atom;
or X⁵ is taken together with X^{5a} and the carbon atom to which they are attached and form a partially saturated or fully saturated 3- to 7-membered ring, or a partially saturated or fully saturated 4- to 8-membered ring having 1 to 4 heteroatoms independently selected from the group consisting of oxygen, sulfur and nitrogen;
or X⁵ is taken together with X^{5a} and the carbon atom to which they are attached and form a bicyclic ring system consisting of a partially saturated or fully saturated 5- or 6-membered ring, optionally having 1 or 2 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
Z¹ is a bond, O or N—X², provided that when a and b are both 0 then Z¹ is not N—X² or O; or
R⁶ is —(CR^{a}R^{b})ₐ-E-(CR^{a}R^{b})_{b}—, where the —(CR^{a}R^{b})ₐ— group is attached to the carbonyl carbon of the amide group of the compound of formula I and the —(CR^{a}R^{b})_{b} group is attached to the terminal nitrogen atom of the compound of formula I;
E is —O—, —S—, —CH═CH— or an aromatic moiety selected from 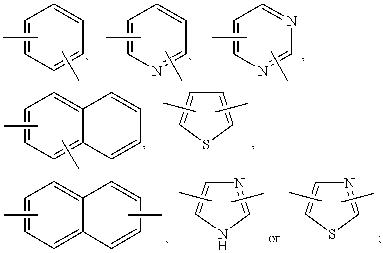
said aromatic moiety in the definition of E optionally substituted with up to three halo, hydroxy, —N(R^{c})(R^{c}), (C₁-C₆)alkyl or (C₁-C₆)alkoxy;
R^{a} and R^{b} are, for each occurrence, independently hydrogen, (C₁-C₆)alkyl, trifluoromethyl, phenyl or monosubstituted (C₁-C₆)alkyl where the substituents are imidazolyl, naphthyl, phenyl, indolyl, p-hydroxyphenyl, —OR^{c}, S(O)ₘR^{c}, C(O)OR^{c}, (C₃-C₇)cycloalkyl, —N(R^{c})(R^{c}), —C(O)N(R^{c})(R^{c}), or Ra or R^{b} may independently be joined to one or both of R⁷or E (where E is other than O, S or —CH═CH—) to form an alkylene bridge between the terminal nitrogen and the alkyl portion of the R^{a} or R^{b} and the R⁷or E group, wherein the bridge contains 1 to 8 carbon atoms; or R^{a} and R^{b} may be joined to one another to form a (C₃-C₇)cycloalkyl;
R^{c}, for each occurrence, is independently hydrogen or (C₁-C₆)alkyl;
a and b are independently 0, 1, 2 or 3, with the proviso that if E is —O— or —S—, b is other than 0 or 1 and with the further proviso that if E is —CH═CH—, b is other than 0;
R⁷ and R⁸ are each independently hydrogen or optionally substituted (C₁-C₆)alkyl;
where the optionally substituted (C₁-C₆)alkyl in the definition of R⁷ and R⁸ is optionally independently substituted with A¹, —C(O)O—(C₁-C₆)alkyl, —S(O)ₘ(C₁-C₆)alkyl, 1 to 5 halo groups, 1 to 3 hydroxy groups, 1 to 3 —O—C(O)(C₁-C₁₀)alkyl groups or 1 to 3 (C₁-C₆)alkoxy groups; or
R⁷ and R⁸ can be taken together to form —(CH₂)ᵣ-L-(CH₂)ᵣ—;
where L is C(X²)(X²), S(O)ₘ or N(X²);
R⁹ and R¹⁰ are each independently selected from the group consisting of hydrogen, fluoro, hydroxy and (C₁-C₅)alkyl optionally independently substituted with 1-5 halo groups;
R¹¹ is selected from the group consisting of (C₁-C₅)alkyl and phenyl optionally substituted with 1-3 substitutents each independently selected from the group consisting of (C₁-C₅)alkyl, halo and (C₁-C₅)alkoxy;
R¹² is selected from the group consisting of (C₁-C₅)alkylsulfonyl, (C₁-C₅)alkanoyl and (C₁-C₅)alkyl where the alkyl portion is optionally independently substituted by 1-5 halo groups;
A¹ for each occurrence is independently selected from the group consisting of (C₅-C₇)cycloalkenyl, phenyl, a partially saturated, fully saturated or fully unsaturated 4- to 8-membered ring optionally having 1 to 4 heteroatoms independently selected from the group consisting of oxygen, sulfur and nitrogen and a bicyclic ring system consisting of a partially saturated, fully unsaturated or fully saturated 5- or 6-membered ring, optionally having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
A¹ for each occurrence is independently optionally substituted, on one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N(X⁶)(X⁶), —N(X⁶)C(O)(X⁶), —S(O)₂N(X⁶)(X⁶), —N(X⁶)S(O)₂-phenyl, —N(X⁶)S(O)₂X⁶, —CONX¹¹X¹², —S(O)₂NX¹¹X¹², —NX⁶S(O)₂X¹², —NX⁶CONX¹¹X¹², —NX⁶S(O)₂NX¹¹X¹², —NX⁶C(O)X¹², imidazolyl, thiazolyl and tetrazolyl, provided that if A¹ is optionally substituted with methylenedioxy then it can only be substituted with one methylenedioxy;
where X¹¹ is hydrogen or optionally substituted (C₁-C₆)alkyl;
the optionally substituted (C₁-C₆)alkyl defined for X¹¹ is optionally independently substituted with phenyl, phenoxy, (C₁-C₆)alkoxycarbonyl, —S(O)ₘ(C₁-C₆)alkyl, 1 to 5 halo groups, 1 to 3 hydroxy groups, 1 to 3 (C₁-C₁₀)alkanoyloxy groups or 1 to 3 (C₁-C₆)alkoxy groups;
X¹² is hydrogen, (C₁-C₆)alkyl, phenyl, thiazolyl, imidazolyl, furyl or thienyl, provided that when X¹² is not hydrogen, the X¹² group is optionally substituted with one to three substituents independently selected from the group consisting of Cl, F, CH₃, OCH₃, OCF₃ and CF₃;
or X¹¹ and X¹² are taken together to form —(CH₂)ᵣ-L-(CH₂)ᵣ—;
L is C(X²)(X²), O, S(O)ₘ or N(X²);
r for each occurrence is independently 1, 2 or 3;
X²for each occurrence is independently hydrogen, optionally substituted (C₁-C₆)alkyl or optionally substituted (C₃-C₇)cycloalkyl, where the optionally substituted (C₁-C₆)alkyl and optionally substituted (C₃-C₇)cycloalkyl in the definition Of X are optionally independently substituted with —S(O)ₘ(C₁-C₆)alkyl, —C(O)OX^{3,} 1 to 5 halo groups or 1-3 OX³ groups;
X³ for each occurrence is independently hydrogen or (C₁-C₆)alkyl;
X⁶ for each occurrence is independently hydrogen, optionally substituted (C₁-C₆)alkyl, (C₂-C₆)halogenated alkyl, optionally substituted (C₃-C₇)cycloalkyl, (C₃-C₇)halogenated cycloalkyl, where optionally substituted (C₁-C₆)alkyl and optionally substituted (C₃-C₇)cycloalkyl in the definition of X⁶is optionally independently mono- or di-substituted with (C₁-C₄)alkyl, hydroxy, (C₁-C₄)alkoxy, carboxyl, CONH₂, —S(O)ₘ(C₁-C₆)alkyl, carboxylate (C₁-C₄)alkyl ester or 1H-tetrazol-5-yl; or when there are two X⁶ groups on one atom and both X⁶ are independently (C₁-C₆)alkyl, the two (C₁-C₆)alkyl groups may be optionally joined and, together with the atom to which the two X⁶groups are attached, form a 4- to 9-membered ring optionally having oxygen, sulfur or NX⁷ as a ring member;
X⁷ is hydrogen or (C₁-C₆)alkyl optionally substituted with hydroxy;
m for each occurrence is independently 0, 1 or 2;
with the proviso that:
X⁶ and X¹² cannot be hydrogen when attached to C(O) or S(O)₂in the form C(O)X⁶, C(O)X¹², S(O)₂X⁶ or S(O)₂X¹²; and
when R⁶ is a bond then L is N(X²) and each r in the definition —(CH₂)ᵣ-L-(CH₂)ᵣ— is independently 2 or 3;
a prodrug thereof or a pharmaceutically acceptable salt of said GHS or of said prodrug.

7. A combination of claim 6 wherein said GHS is a compound of the formula 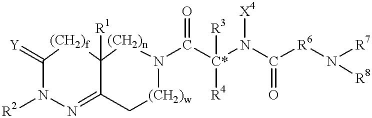
or a stereoisomeric mixture thereof, diastereomerically enriched, diastereomerically pure, enantiomerically enriched or enantiomerically pure isomer thereof,
wherein:
f is 0;
n is 0 and w is 2, or n is 1 and w is 1, or n is 2 and w is 0;
Y is oxygen or sulfur;
R¹ is hydrogen, —CN, —(CH₂)_{q}N(X⁶)C(O)X⁶, —(CH₂)_{q}N(X⁶)C(O)(CH₂)ₜ-A¹, (CH₂)_{q}N(X⁶)SO₂(CH₂)ₜ-A¹, (CH₂)_{q}N(X⁶)SO₂X⁶, (CH₂)_{q}N(X⁶)C(O)N(X⁶)(CH₂)ₜ-A¹, —(CH₂)_{q}N(X⁶)C(O)N(X⁶)(X⁶), —(CH₂)_{q}C(O)N(X⁶)(X⁶), —(CH₂)_{q}C(O)N(X⁶)(CH₂)ₜ-A¹, —(CH₂)_{q}C(O)OX⁶, (CH₂)_{q}C(O)O(CH₂)ₜA¹(CH₂)_{q}OX⁶, (CH₂)_{q}OC(O)X⁶, (CH₂)_{q}OC(O)(CH₂)ₜ-A¹, —(CH₂)_{q}OC(O)N(X⁶)(CH₂)ₜ-A¹l, (CH₂)_{q}OC(O)N(X⁶)(X⁶), (CH₂)_{q}C(O)X⁶, —(CH₂)_{q}C(O)(CH₂)ₜ-A¹, (CH₂)_{q}N(X⁶)C(O)OX⁶, —(CH₂)_{q}N (X⁶)SO₂N(X⁶)(X⁶), (CH₂)_{q}S(O)ₘX⁶, (CH₂)_{q}S(O)ₘ(CH₂)ₜ-A¹, —(C₁-C₁₀)alkyl, —(CH₂)ₜ-A¹, —(CH₂)_{q}—(C₃-C₇)cycloalkyl, —(CH₂)_{q}—Y¹—(C₁-C₆)alkyl, (CH₂)_{q}Y¹—(CH₂)ₜ-A¹or —(CH₂)_{q}—Y¹—(CH₂)t—(C₃-C₇)cycloalkyl;
where the alkyl and cycloalkyl groups in the definition of R¹ are optionally substituted with (C₁-C₄)alkyl, hydroxyl, (C₁-C₄)alkoxy, carboxyl, —CONH₂, —S(O)ₘ(C₁-C₆)alkyl, —CO₂(C₁-C₄)alkyl ester, 1H-tetrazol-5-yl or 1, 2 or 3 fluoro;
Y¹ is 0, S(O)ₘ, —C(O)NX⁶—, —CH═CH—, —C≡C—, —N(X⁶)C(O)—, —C(O)NX⁶—, —C(O)O—, —OC(O)N(X⁶)— or —OC(O)—;
q is 0, 1, 2, 3 or 4;
t is 0, 1, 2 or 3;
said (CH₂)_{q} group and (CH₂)ₜ group may each be optionally substituted with hydroxyl, (C₁-C₄)alkoxy, carboxyl, —CONH₂, —S(O)ₘ(C₁-C₆)alkyl, —CO₂(C₁-C₄)alkyl ester, 1H-tetrazol-5-yl, 1, 2 or 3 fluoro, or 1 or 2 (C₁-C₄)alkyl;
R² is hydrogen, (C₁-C₈)alkyl, —(C₀-C₃)alkyl-(C₃-C₈)cycloalkyl, —(C₁-C₄)alkyl-A¹ or A¹;
where the alkyl groups and the cycloalkyl groups in the definition of R² are optionally substituted with hydroxyl, —C(O)OX⁶, —C(O)N(X⁶)(X⁶), —N(X⁶)(X⁶), —S(O)ₘ(C₁-C₆)alkyl, —C(O)A¹, —C(O)(X⁶), CF₃, CN or 1, 2 or 3 halogen;
R³ is A¹, (C₁-C₁₀)alkyl, —(C₁-C₆)alkyl-A¹, —(C₁-C₆)alkyl-(C₃-C₇)cycloalkyl, —(C₁-C₅)alkyl-X¹—(C₁-C₅)alkyl, —(C₁-C₅)alkyl-X¹—(C₀-C₅)alkyl-A¹ or —(C₁-C₅)alkyl-X—(C₁-C₅)alkyl-(C₃-C₇)cycloalkyl;
where the alkyl groups in the definition of R³ are optionally substituted with, —S(O)ₘ(C₁-C₆)alkyl, —C(O)OX³, 1, 2, 3, 4 or 5 halogens, or 1, 2 or 3 Ox³;
X¹ is O, S(O)ₘ, —N(X²)C(O)—, —C(O)N(X²)—, —OC(O)—, —C(O)O—, —CX²═CX²—, —N(X²)C(O)O—, —OC(O)N(X²)— or —C≡C—;
R⁴ is hydrogen, (C₁-C₆)alkyl or (C₃-C₇ )cycloalkyl;
X⁴ is hydrogen or (C₁-C₆ )alkyl or X⁴ is taken together with R⁴ and the nitrogen atom to which X⁴ is attached and the carbon atom to which R⁴ is attached and form a five to seven membered ring;
R⁶ is a bond or is 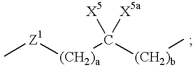
where a and b are independently 0, 1, 2 or 3;
X⁵ and X^{5a} are each independently selected from the group consisting of hydrogen, trifluoromethyl, A¹ and optionally substituted (C₁-C₆)alkyl;
the optionally substituted (C₁-C₆)alkyl in the definition of X⁵ and X^{5a} is optionally substituted with a substituent selected from the group consisting of A¹, OX², —S(O)ₘ(C₁-C₆)alkyl, —C(O)OX², (C₃-C₇)cycloalkyl, —N(X²)(X²) and —C(O)N(X²)(X²);
R⁷ and R⁸ are independently hydrogen or optionally substituted (C₁-C₆)alkyl;
where the optionally substituted (C₁-C₆)alkyl in the definition of R⁷ and R⁸ is optionally independently substituted with A¹, —C(O)O—(C₁-C₆)alkyl, —S(O)ₘ(C₁-C₆)alkyl, 1 to 5 halogens, 1 to 3 hydroxy, 1 to 3 —O—C(O)(C₁-C₁₀)alkyl or 1 to 3 (C₁-C₆)alkoxy; or
R⁷ and R⁸ can be taken together to form —(CH₂)ᵣ-L-(CH₂)ᵣ—;
where L is C(X²)(X²), S(O)ₘ or N(X²);
A¹ in the definition of R¹ is a partially saturated, fully saturated or fully unsaturated 4- to 8-membered ring optionally having 1 to 4 heteroatoms independently selected from the group consisting of oxygen, sulfur and nitrogen, a bicyclic ring system consisting of a partially saturated, fully unsaturated or fully saturated 5- or 6-membered ring, having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen; A¹ in the definition of R², R³, R⁶, R⁷ and R⁸ is independently (C₅-C₇)cycloalkenyl, phenyl or a partially saturated, fully saturated or fully unsaturated 4- to 8-membered ring optionally having 1 to 4 heteroatoms independently selected from the group consisting of oxygen, sulfur and nitrogen, a bicyclic ring system consisting of a partially saturated, fully unsaturated or fully saturated 5- or 6-membered ring, optionally having 1 to 4 heteroatoms independently selected from the group consisting of nitorgen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
A¹ for each occurrence is independently optionally substituted, in one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N(X⁶)(X⁶), —N(X⁶)C(O)(X⁶), —SO₂ N(X⁶)(X⁶), —N(X⁶)SO₂-phenyl, —N(X⁶)SO₂ X⁶, —CONX¹¹X¹², —SO₂NX¹¹X¹², —NX⁶ SO₂ X¹², —NX⁶CONX¹¹X¹², —NX⁶SO₂NX¹¹X¹², —NX⁶C(O)X¹², imidazolyl, thiazolyl or tetrazolyl, provided that if A¹ is optionally substituted with methylenedioxy then it can only be substituted with one methylenedioxy;
where X¹¹ is hydrogen or optionally substituted (C₁-C₆)alkyl;
the optionally substituted (C₁-C₆)alkyl defined for X¹¹ is optionally independently substituted with phenyl, phenoxy, (C₁-C₆)alkoxycarbonyl, —S(O)ₘ(C₁-C₆)alkyl 1 to 5 halogens, 1 to 3 hydroxy, 1 to 3 (C₁-C₁₀)alkanoyloxy or 1 to 3 (C₁-C₆)alkoxy;
X¹² is hydrogen, (C₁-C₆)alkyl, phenyl, thiazolyl, imidazolyl, furyl or thienyl, provided that when X¹² is not hydrogen, X¹² is optionally substituted with one to three substituents independently selected from the group consisting of Cl, F, CH₃, OCH₃, OCF₃ and CF₃;
or X¹¹ and X¹² are taken together to form —(CH₂)ᵣ-L¹-(CH₂)ᵣ;
where L¹ is C(X²)(X²), O, S(O)ₘ or N(X²);
r for each occurrence is independently 1, 2 or 3;
X² for each occurrence is independently hydrogen, optionally substituted (C₁-C₆)alkyl, or optionally substituted (C₃-C₇)cycloalkyl, where the optionally substituted (C₁-C₆)alkyl and optionally substituted (C₃-C₇)cycloalkyl in the definition of X² are optionally independently substituted with —S(O)ₘ(C₁-C₆)alkyl, —C(O)OX³, 1 to 5 halogens or 1-3 OX³;
X³ for each occurrence is independently hydrogen or (C₁-C₆)alkyl;
X⁶ is independently hydrogen, optionally substituted (C₁-C₆)alkyl, (C₂-C₆)halogenated alkyl, optionally substituted (C₃-C₇)cycloalkyl, (C₃-C₇)-halogenatedcycloalkyl, where optionally substituted (C₁-C₆)alkyl and optionally substituted (C₃-C₇)cycloalkyl in the definition of X⁶ is optionally independently substituted by 1 or 2 (C₁-C₄)alkyl, hydroxyl, (C₁-C₄)alkoxy, carboxyl, CONH₂, —S(O)ₘ(C₁-C₆)alkyl, carboxylate (C₁-C₄)alkyl ester, or 1H-tetrazol-5-yl; or
when there are two X⁶ groups on one atom and both X⁶ are independently (C₁-C₆)alkyl, the two (C₁-C₆)alkyl groups may be optionally joined and, together with the atom to which the two X⁶ groups are attached, form a 4- to 9-membered ring optionally having oxygen, sulfur or NX⁷;
X⁷ is hydrogen or (C₁-C₆)alkyl optionally substituted with hydroxyl; and
m for each occurrence is independently 0, 1 or 2;
with the proviso that:
X⁶ and X¹² cannot be hydrogen when it is attached to C(O) or SO₂ in the form C(O)X⁶, C(O)X¹², SO₂X⁶ or SO₂X¹²; and
when R⁶ is a bond then L is N(X²) and each r in the definition —(CH₂)ᵣ-L-(CH₂)ᵣ— is independently 2 or 3.

8. A combination of claim 7 wherein said GHS is 2-amino-N-(1(R)-benzyloxymethyl-2-(1,3-dioxo-8a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide or a pharmaceutically acceptable salt thereof.

9. A combination of claim 8 wherein said GHS is the L-(+)-tartaric acid salt of 2-amino-N-(1(R)-benzyloxymethyl-2-(1,3-dioxo-[a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide.

10. A combination of claim 7 wherein said GHS is 2-amino-N-[2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide or a pharmaceutically acceptable salt thereof.

11. A combination of claim 10 wherein said GHS is the 2-amino-N-[2-(3 a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide, L-tartrate.

12. A combination of claim 7 wherein said GHS is 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo[4,3-c]pyridin-5-yl)-ethyl)-2-methyl-propionamide, or a pharmaceutically acceptable salt thereof.

13. A combination of claim 12 wherein said GHS is the L-(+)-tartaric acid salt of 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo[4,3-c]pyridin-5-yl)-ethyl)-2-methyl-propionamide.

14. A combination of claim 1 wherein said GHS is hexarelin, ipamorelin, MK-0677, NN703, L-162752, L-163022, GPA-748, KP102, GHRP-2 or LY444711.

15. A combination of claim 5 wherein said GHS is 2-amino-N-(1 (R)-benzyloxymethyl-2-(1,3-dioxo-8a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide or a pharmaceutically acceptable salt thereof.

16. A combination of claim 15 wherein said GHS is the L-(+)-tartaric acid salt of 2-amino-N-(1(R)-benzyloxymethyl-2-(1,3-dioxo-8a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide and said SSRI is sertraline hydrochloride.

17. A combination of claim 5 wherein said GHS is 2-amino-N-[2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide or a pharmaceutically acceptable salt thereof.

18. A combination of claim 17 wherein said GHS is the 2-amino-N-[2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide, L-tartrate and said SSRI is sertraline hydrochloride.

19. A combination of claim 5 wherein said GHS is 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo[4,3-c]pyridin-5-yl)-ethyl)-2-methyl-propionamide, or a pharmaceutically acceptable salt thereof.

20. A combination of claim 19 wherein said GHS is the L-(+)-tartaric acid salt of 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo[4,3-c]pyridin-5-yl)-ethyl)-2-methyl-propionamide and said SSRI is sertraline hydrochloride.

21. A pharmaceutical composition comprising a combination of claim 1 and a pharmaceutically acceptable carrier, vehicle or diluent.

22. A method of improving the physical or psychological condition of a patient undergoing a medical procedure comprising administering to said patient:
a) a pharmaceutical composition of claim 21; or
b) a combination of a growth hormone secretagogue (GHS), prodrug thereof, pharmaceutically acceptable salt of said GHS or of said prodrug or a pharmaceutical composition thereof and an antidepressant, a prodrug thereof, pharmaceutically acceptable salt of said antidepressant or said prodrug or a pharmaceutical composition thereof.

23. A method of claim 22 wherein said physical condition is the cardiac function of said patient.

24. A method of claim 22 wherein said physical condition is the metabolism of said patient.

25. A method of claim wherein said physical condition is the muscle tone of said patient.

26. A method of claim 22 wherein said physical condition is the mental state of said patient.

27. A method of claim 22 wherein said medical procedure is a surgical or dental procedure.

28. A method of claim 27 wherein said combination or pharmaceutical composition is administered prior to said surgical or dental procedure.

29. A method of claim 27 wherein said combination or pharmaceutical composition is administered during said surgical or dental procedure.

30. A method of claim 27 wherein said combination or pharmaceutical composition is administered after said surgical or dental procedure.

31. A method of claim 22 wherein said antidepressant is an SSRI, a prodrug thereof or a pharmaceutically acceptable salt of said SSRI or said prodrug.

32. A method of claim 31 wherein said SSRI is femoxetine, fluoxetine, fluvoxamine, indalpine, indeloxazine, milnacipran, paroxetine, sertraline, sibutramine or zimeldine, a prodrug thereof or a pharmaceutically acceptable salt of said SSRI or of said prodrug.

33. A method of claim 32 wherein said SSRI is fluoxetine, sertraline or sibutramine, a prodrug thereof or a pharmaceutically acceptable salt of said SSRI or said prodrug.

34. A method of claim 22 wherein said GHS is a compound of the formula I: 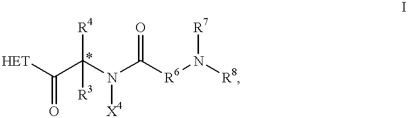
or a stereoisomeric mixture thereof, diastereomerically enriched, diastereomerically pure, enantiomerically enriched or enantiomerically pure isomer thereof,
wherein:
HET is a heterocyclic moiety selected from the group consisting of [chemical structure]
d is 0, 1 or 2;
e is 1 or 2;
f is 0 or 1;
n and w are 0, 1 or 2, provided that n and w cannot both be 0 at the same time;
Y² is oxygen or sulfur;
A is a divalent radical, where the left hand side of the radical as shown below is connected to C′ and the right hand side of the radical as shown below is connected to C′, selected from the group consisting of
—NR²—C(O)—NR²—, —NR²—S(O)NR²—, —O—C(O)—NR²—, —NR²—C(O)—O—, —C(O)—NR²—C(O)—, —C(O)—NR²—C(R⁹R¹⁰)—, —C(R⁹R¹⁰)—NR²—C(O)—, —C(R⁹R¹⁰)—C(R⁹R¹⁰)—C(R⁹R¹⁰), —S(O)₂—C(R⁹R¹⁰)—C(R⁹R¹⁰)—, —C(R⁹R¹⁰)—O—C(O)—, —C(R⁹R¹⁰)—O—C(R⁹R¹⁰)—, —NR²—C(O)—C(R⁹R¹⁰)—, —O—C(O)—C(R⁹R¹⁰)—, —C(R⁹R¹⁰)—C(O)—NR²—, —C(O)—NR²—C(O)—, —C(R⁹R¹⁰)—C(O)—O—, —C(O)—NR²—C(R⁹R¹⁰)—C(R⁹R¹⁰)—, —C(O)—O—C(R⁹R¹⁰)—, —C(R⁹R¹⁰)—C(R⁹R¹⁰)—C(R⁹R¹⁰)—C(R⁹R¹⁰)—, —S(O)₂—NR—C(R⁹R¹⁰)—C(R⁹R¹⁰)—, —C(R⁹R¹⁰)—C(R⁹R¹⁰)—N R²—C(O)—, —C(R⁹R¹⁰)—C(R⁹R¹⁰)—O—C(O)—, —N R²—C(O)—C(R⁹R¹)—C(R⁹R¹⁰)—, —N R²—S(O)₂ —C(R⁹R¹⁰)—C(R⁹R¹⁰)—, —O—C(O)—C(R⁹R¹⁰)—C(R⁹R¹⁰), —C(R⁹R¹⁰)—C(R⁹R¹⁰)—C(O)—NR²—, —C(R⁹R¹⁰)—C(R⁹R¹⁰)—C(O)—, —C(R⁹R¹⁰)—N R²—C(O)—O—, —C(R⁹R¹⁰)—O—C(O)—NR², —C(R⁹R¹⁰)—NR²—C(O)—NR²—, —NR²—C(O)—O—C(R⁹R¹⁰)—, —NR²—C(O)—NR²—C(R⁹R¹⁰)—, —NR²—S(O)₂—NR²—C(R⁹R¹⁰)—, —O—C(O)—NR²—C(R⁹R¹⁰)—, —C(O)—N═C(R¹¹)—NR²—, —C(O)—NR —C(R¹¹)═N—, —C(R⁹R¹⁰)—NR¹² C(R⁹R¹⁰)—, —NR^{12C(R}⁹R¹⁰)—, —NR^{12C(R}⁹R¹⁰)—C(R⁹R¹⁰)—, —C(O)—O—C(R⁹R¹⁰)—C(R⁹R¹⁰)—, —NR¹² —C(R¹¹)═N—C(O)—, —C(R⁹R¹⁰)—C(R⁹R¹⁰)—N(R¹²)—, —C(R⁹R¹⁰)—NR^{12-, —N═C(R}¹¹)—NR²—C(O)—, —C(R⁹R¹⁰)—C(R⁹R¹⁰)—NR²—S(O)₂—, —C(R⁹R¹⁰)—C(R⁹R¹⁰)—S(O)₂—NR²—, —C(R⁹R¹⁰)—C(R⁹R¹⁰)—C(O)—O—, —C(R⁹R¹⁰)—S(O)₂—C(R⁹R¹⁰)—, —C(R⁹R¹⁰)—C(R⁹R¹)—S(O)₂—, —O—C(R⁹R¹⁰)—C(R⁹R¹⁰)—, —C(R⁹R¹⁰)—C(R⁹R¹⁰)—O—, —C(R⁹R¹⁰)—C(O)—C(R⁹R¹⁰)—, —C(O)—C(R⁹R¹⁰)—C(R⁹R¹⁰)— and —C(R⁹R¹⁰)—NR²—S(O)₂—NR²—;
Q is a covalent bond or CH₂;
W is CH or N;
X is CR⁹R¹⁰, C═CH₂ or C═O;
Y is CR⁹R¹⁰, O or NR²;
Z is C═O, C═S or S(O)₂;
G¹ is hydrogen, halo, hydroxy, nitro, amino, cyano, phenyl, carboxyl, —CONH₂, —(C₁-C₄)alkyl optionally independently substituted with one or more phenyl, one or more halogens or one or more hydroxy groups, —(C₁-C₄)alkoxy optionally independently substituted with one or more phenyl, one or more halogens or one or more hydroxy groups, —(C₁-C₄)alkylthio, phenoxy, —COO(C₁-C₄)alkyl, N,N-di-(C₁-C₄)alkylamino, —(C₂-C₆)alkenyl optionally independently substituted with one or more phenyl, one or more halogens or one or more hydroxy groups, —(C₂-C₆)alkynyl optionally independently substituted with one or more phenyl, one or more halogens or one or more hydroxy groups, —(C₃-C₆)cycloalkyl optionally independently substituted with one or more (C₁-C₄)alkyl groups, one or more halogens or one or more hydroxy groups, —(C₁-C₄)alkylamino carbonyl or di-(C₁-C₄)alkylamino carbonyl;
G² and G³ are each independently selected from the group consisting of hydrogen, halo, hydroxy, —(C₁-C₄)alkyl optionally independently substituted with one to three halo groups and —(C₁-C₄)alkoxy optionally independently substituted with one to three halo groups;
R¹ is hydrogen, —CN, —(CH₂)_{q}N(X⁶)C(O)X⁶, —(CH₂)_{q}N(X⁶)C(O)(CH₂)ₜ-A¹, (CH₂)_{q}N(X⁶)S(O)₂ (CH₂)ₜA¹, —(CH₂)_{q}N(X⁶)S(O)₂ X⁶, —(CH₂)_{q}N(X⁶)C(O)N(X⁶)(CH₂)ₜA¹, (CH₂)_{q}N(X⁶)C(O)N(X⁶)(X⁶), (CH₂)_{q}C(O)N(X⁶)(X⁶), (CH₂)_{q}C(O)N(X⁶)(CH₂)ₜA¹, (CH₂)_{q}C(O)OX⁶, —(CH₂)_{q}C(O)O(CH₂)ₜ-A¹, (CH₂)_{q}OX⁶, (CH₂)_{q}OC(O)X⁶, (CH₂)_{q}OC(O)(CH₂)ₜA¹, (CH₂)_{q}OC(O)N(X⁶)(CH₂)ₜA¹, (CH₂)_{q}OC(O)N (X⁶)(X⁶), —(CH₂)_{q}C(O)X⁶, —(CH₂)_{q}C(O)(CH₂)ₜA¹, (CH₂)_{q}N(X⁶)C(O)OX⁶, (CH₂)_{q}N(X⁶)S(O)₂ N(X⁶)(X⁶), (CH₂)_{q}S(O)ₘX⁶, —(CH₂)_{q}S(O)ₘ(CH₂)ₜ-A¹, —(C₁-C₁₀)alkyl, —(CH₂)ₜ-A¹, —(CH₂)_{q}—(C₃-C₇)cycloalkyl, (CH₂)_{q}Y¹ —(C₁-C₆)alkyl, (CH₂ 2)_{q}Y¹ —(CH₂)ₜ-A¹ or —(CH₂)_{q}—Yl—(CH₂)ₜ —(C₃-C₇)cycloalkyl;
where the alkyl and cycloalkyl groups in the definition of R¹ are optionally substituted with (C₁-C₄)alkyl, hydroxy, (C₁-C₄)alkoxy, carboxyl, —CONH₂, —S(O)ₘ(C₁-C₆)alkyl, —CO₂(C₁-C₄)alkyl ester, 1H-tetrazol-5-yl or 1, 2 or 3 fluoro groups;
Y¹ is O, S(O)ₘ, —C(O)NX⁶—, —CH═CH—, —C≡C—, —N(X⁶)C(O)—, —C(O)NX⁶—, —C(O)O—, —OC(O)N(X⁶)— or —OC(O)—;
q is 0, 1, 2, 3 or 4;
t is 0, 1, 2 or 3;
said (CH₂)_{q}group and (CH₂)ₜ group in the definition of R¹ are optionally independently substituted with hydroxy, (C₁-C₄)alkoxy, carboxyl, —CONH₂, —S(O)ₘ(C₁-C₆)alkyl, —CO₂(C₁-C₄)alkyl ester, 1H-tetrazol-5-yl, 1, 2 or 3 fluoro groups or 1 or 2 (C₁-C₄)alkyl groups;
R^{1A} is selected from the group consisting of hydrogen, F, Cl, Br, I, (C₁-C₆)alkyl, phenyl(C₁-C₃)alkyl, pyridyl(C₁-C₃)alkyl, thiazolyl(C₁-C₃)alkyl and thienyl(C₁-C₃)alkyl, provided that R^{1A} is not F, Cl, Br or I when a heteroatom is vicinal to C″;
R² is hydrogen, (C₁-C₈)alkyl, —(C₀-C₃)alkyl-(C₃-C₈)cycloalkyl, —(C₁-C₄)alkyl-A¹ or A¹;
where the alkyl groups and the cycloalkyl groups in the definition of R²are optionally substituted with hydroxy, —C(O)OX⁶, —C(O)N(X⁶)(X⁶), —N (X⁶)(X⁶), —S(O)ₘ(C₁-C₆)alkyl, —C(O)A¹, —C(O)(X⁶), CF₃, CN or 1, 2 or 3 independently selected halo groups;
R³ is selected from the group consisting of A¹, (C₁-C₁₀)alkyl, —(C₁-C₆)alkyl-A¹, —(C₁-C₆)alkyl-(C₃-C₇)cycloalkyl, —(C₁-C₅)alkyl-X—(C₁-C₅)alkyl, —(C₁-C₅)alkyl-X—(C₀-C₅)alkyl-A¹ and —(C₁-C₅)alkyl-X¹—(C₁-C₅)alkyl-(C₃-C₇)cycloalkyl;
where the alkyl groups in the definition of R³ are optionally substituted with —S(O)ₘ(C₁-C₆)alkyl, —C(O)OX³, 1, 2, 3, 4 or 5 independently selected halo groups or 1, 2 or 3 independently selected —OX³ groups;
X¹ is S(O)ₘ, —N(X²)C(O)—, —C(O)N(X²)—, —OC(O)—, —C(O)O—, —CX²═CX²—, —N(X²)C(O)O—, —OC(O)N(X²)— or —C≡C—;
R⁴ is hydrogen, (C₁-C₆)alkyl or (C₃-C₇)cycloalkyl, or R⁴ is taken together with R³ and the carbon atom to which they are attached and form (C₅-C₇)cycloalkyl, (C₅-C₇)cycloalkenyl, a partially saturated or fully saturated 4- to 8-membered ring having 1 to 4 heteroatoms independently selected from the group consisting of oxygen, sulfur and nitrogen, or is a bicyclic ring system consisting of a partially saturated or fully saturated 5- or 6-membered ring, fused to a partially saturated, fully unsaturated or fully saturated 5- or 6-membered ring, optionally having 1 to 4 heteroatoms independently selected from the group consisting of nitrogen, sulfur and oxygen;
X⁴ is hydrogen or (C₁-C₆)alkyl or X⁴ is taken together with R⁴and the nitrogen atom to which X⁴ is attached and the carbon atom to which R⁴ is attached and form a five to seven membered ring;
R⁶ is a bond or is 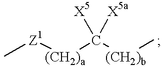
X⁵ and X^{5a} are each independently selected from the group consisting of hydrogen, CF₃, A¹ and optionally substituted (C₁-C₆)alkyl;
the optionally substituted (C₁-C₆)alkyl in the definition of X⁵ and X^{5a} is optionally substituted with a substituent selected from the group consisting of A¹, OX², —S(O)ₘ(C₁-C₆)alkyl, —C(O)0X², (C₃-C₇)cycloalkyl, —N(X²)(X²) and —C(O)N(X²)(X²);
or the carbon bearing X⁵ or X^{5a} forms one or two alkylene bridges with the nitrogen atom bearing R⁷ and R⁸ wherein each alkylene bridge contains 1 to 5 carbon atoms, provided that when one alkylene bridge is formed then only one of X⁵ or X^{5a} is on the carbon atom and only one of R⁷ or R⁸ is on the nitrogen atom and further provided that when two alkylene bridges are formed then X⁵ and X^{5a} cannot be on the carbon atom and R⁷and R⁸ cannot be on the nitrogen atom;
or X⁵ is taken together with X^{5a} and the carbon atom to which they are attached and form a partially saturated or fully saturated 3- to 7-membered ring, or a partially saturated or fully saturated 4- to 8-membered ring having 1 to 4 heteroatoms independently selected from the group consisting of oxygen, sulfur and nitrogen;
or X⁵ is taken together with X^{5a} and the carbon atom to which they are attached and form a bicyclic ring system consisting of a partially saturated or fully saturated 5- or 6-membered ring, optionally having 1 or 2 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
Z¹ is a bond, O or N—X², provided that when a and b are both 0 then Z¹ is not N—X² or O; or
R⁶ is —(CR^{a}R^{b})ₐ-E-(CR^{a}R^{b})_{b}—, where the —(CR^{a} R^{b})ₐ— group is attached to the carbonyl carbon of the amide group of the compound of formula I and the —(CR^{a} R^{b})_{b} group is attached to the terminal nitrogen atom of the compound of formula I;
E is —O—, —S—, —CH═CH— or an aromatic moiety selected from 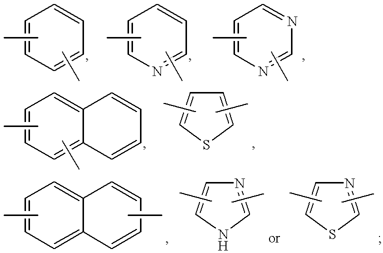
said aromatic moiety in the definition of E optionally substituted with up to three halo, hydroxy, —N(R^{c})(R^{c}), (C₁-C₆)alkyl or (C₁-C₆)alkoxy;
R^{a} and R^{b} are, for each occurrence, independently hydrogen, (C₁-C₆)alkyl, trifluoromethyl, phenyl or monosubstituted (C₁-C₆)alkyl where the substituents are imidazolyl, naphthyl, phenyl, indolyl, p-hydroxyphenyl, —OR^{c}, S(O)ₘ R^{c}, C(O)OR^{c}, (C₃-C₇)cycloalkyl, —N(R^{c})(R^{c}), —C(O)N(R^{c})(R^{c}), or R^{a} or R^{b} may independently be joined to one or both of R⁷ or E (where E is other than O, S or —CH═CH—) to form an alkylene bridge between the terminal nitrogen and the alkyl portion of the R^{a} or R^{b}and the R⁷ or E group, wherein the bridge contains 1 to 8 carbon atoms; or R^{a}and R^{b} may be joined to one another to form a (C₃-C₇)cycloalkyl;
R^{c}, for each occurrence, is independently hydrogen or (C₁-C₆)alkyl;
a and b are independently 0, 1, 2 or 3, with the proviso that if E is —O- or —S—, b is other than 0 or 1 and with the further proviso that if E is —CH═CH—, b is other than 0;
R⁷ and R⁸ are each independently hydrogen or optionally substituted (C₁-C₆)alkyl;
where the optionally substituted (C₁-C₆)alkyl in the definition of R⁷ and R⁸ is optionally independently substituted with A¹, —C(O)O—(C₁-C₆)alkyl, —S(O)ₘ(C₁-C₆)alkyl, 1 to 5 halo groups, 1 to 3 hydroxy groups, 1 to 3 —O—C(O)(C₁-C₁₀)alkyl groups or 1 to 3 (C₁-C₆)alkoxy groups; or
R⁷and R⁸ can be taken together to form —(CH₂)ᵣ-L-(CH₂)ᵣ—;
where L is C(X²)(X²), S(O)ₘ or N(X²);
R⁹ and R¹⁰ are each independently selected from the group consisting of hydrogen, fluoro, hydroxy and (C₁-C₅)alkyl optionally independently substituted with 1-5 halo groups;
R¹¹ is selected from the group consisting of (C₁-C₆)alkyl and phenyl optionally substituted with 1-3 substitutents each independently selected from the group consisting of (C₁-C₅)alkyl, halo and (C₁-C₅)alkoxy;
R¹² is selected from the group consisting of (C₁-C₅)alkylsulfonyl, (C₁-C₅)alkanoyl and (C₁-C₅)alkyl where the alkyl portion is optionally independently substituted by 1-5 halo groups;
A¹ for each occurrence is independently selected from the group consisting of (C₅-C₇)cycloalkenyl, phenyl, a partially saturated, fully saturated or fully unsaturated 4- to 8-membered ring optionally having 1 to 4 heteroatoms independently selected from the group consisting of oxygen, sulfur and nitrogen and a bicyclic ring system consisting of a partially saturated, fully unsaturated or fully saturated 5- or 6-membered ring, optionally having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
A¹ for each occurrence is independently optionally substituted, on one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N (X⁶)(X⁶), —N(X⁶)C(O)(X⁶), —S(O)₂ N (X⁶)(X⁶), —N(X⁶)S(O)₂-phenyl, —N(X⁶)S(O)₂X⁶, —CONX¹¹X¹², —S(O)₂NX¹¹X¹², —NX⁶S(O)₂X¹², —NX⁶CONX¹¹X¹², —NX⁶S(O)₂NX¹¹X¹², —NX⁶C(O)X¹², imidazolyl, thiazolyl and tetrazolyl, provided that if A¹ is optionally substituted with methylenedioxy then it can only be substituted with one methylenedioxy;
where X¹¹ is hydrogen or optionally substituted (C₁-C₆)alkyl;
the optionally substituted (C₁-C₆)alkyl defined for X¹ is optionally independently substituted with phenyl, phenoxy, (C₁-C₆)alkoxycarbonyl, —S(O)ₘ(C₁-C₆)alkyl, 1 to 5 halo groups, 1 to 3 hydroxy groups, 1 to 3 (C₁-C₁₀)alkanoyloxy groups or 1 to 3 (C₁-C₆)alkoxy groups;
X¹² is hydrogen, (C₁-C₆)alkyl, phenyl, thiazolyl, imidazolyl, furyl or thienyl, provided that when X¹² is not hydrogen, the X¹² group is optionally substituted with one to three substituents independently selected from the group consisting of Cl, F, CH₃, OCH₃, OCF₃and CF₃;
or X¹¹ and X¹² are taken together to form —(CH₂)ᵣ-L¹-(CH₂)ᵣ—;
L¹ is C(X²)(X²), O S(O)ₘ or N(X²);
r for each occurrence is independently 1, 2 or 3;
X² for each occurrence is independently hydrogen, optionally substituted (C₁-C₆)alkyl or optionally substituted (C₃-C₇)cycloalkyl, where the optionally substituted (C₁-C₆)alkyl and optionally substituted (C₃-C₇)cycloalkyl in the definition of X²are optionally independently substituted with —S(O)ₘ(C₁-C₆)alkyl, —C(O)IX³, 1 to 5 halo groups or 1-3 OX³ groups;
X³ for each occurrence is independently hydrogen or (C₁-C₆)alkyl;
X⁶ for each occurrence is independently hydrogen, optionally substituted (C₁-C₆)alkyl, (C₂-C₆)halogenated alkyl, optionally substituted (C₃-C₇)cycloalkyl, (C₃-C₇)-halogenated cycloalkyl, where optionally substituted (C₁-C₆)alkyl and optionally substituted (C₃-C₇)cycloalkyl in the definition of X⁶ is optionally independently mono- or di-substituted with (C₁-C₄)alkyl, hydroxy, (C₁-C₄)alkoxy, carboxyl, CONH₂, —S(O)ₘ(C₁-C₆)alkyl, carboxylate (C₁-C₄)alkyl ester or 1H-tetrazol-5-yl; or when there are two X⁶ groups on one atom and both X⁶ are independently (C₁-C₆)alkyl, the two (C₁-C₆)alkyl groups may be optionally joined and, together with the atom to which the two X⁶ groups are attached, form a 4- to 9-membered ring optionally having oxygen, sulfur or NX⁷ as a ring member;
X⁷ is hydrogen or (C₁-C₆)alkyl optionally substituted with hydroxy;
m for each occurrence is independently 0, 1 or 2;
with the proviso that:
X⁶ and X¹² cannot be hydrogen when attached to C(O) or S(O)₂ in the form C(O)X⁶, C(O)X¹², S(O)₂X⁶ or S(O)₂X¹²; and
when R⁶ is a bond then L is N(X²) and each r in the definition —(CH₂)ᵣ-L-(CH₂)ᵣ- is independently 2 or 3; a prodrug thereof or a pharmaceutically acceptable salt of said GHS or of said prodrug.

35. A method of claim 34 wherein said GHS is a compound of the formula 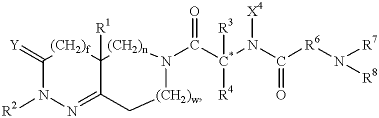
the racemic-diastereomeric mixtures and optical isomers of said compounds and the pharmaceutically-acceptable salts and prodrugs thereof,
wherein
f is 0;
n is 0 and w is 2, or n is 1 and w is 1, or n is 2 and w is 0;
Y is oxygen or sulfur;
R¹ is hydrogen, —CN, —(CH₂)_{q}N(X⁶)C(O)X⁶, —(CH₂)_{q}N(X⁶)C(O)(CH₂)ₜ-A¹, (CH₂)_{q}N(X⁶)SO₂ (CH₂)ₜ-A¹ 7, (CH₂)_{q}N(X⁶)SO₂ X⁶ (CH₂)_{q}N(X⁶)C(O)N(X⁶)(CH₂)ₜ-A¹, (CH₂)_{q}N(X⁶)C(O)N (X⁶)(X⁶), (CH₂)_{q}C(O)N(X⁶)(X⁶), (CH₂)_{q}C(O)N (X⁶)(CH₂)ₜ-A, (CH₂)_{q}C(O)OX⁶, (CH₂)_{q}C(O)O(CH₂)ₜ-A¹ l, (CH₂)_{q}OX⁶, (CH₂)_{q}OC(O)X⁶, —(CH₂)_{q}OC(O)(CH₂)ₜ-A, —(CH₂)_{q}OC(O)N(X⁶)(CH₂)ₜ-A¹, —(CH₂)_{q}OC(O)N (X⁶)(X⁶), (CH₂)_{q}C(O)X⁶, (CH₂)_{q}C(O)(CH₂)ₜ-A¹, (CH₂)_{q}N(X⁶)C(O)OX⁶, (CH₂)_{q}N(X⁶)SO₂ N(X⁶)(X⁶), (CH₂)_{q}S(O)ₘX⁶, —(CH₂)_{q}S(O)ₘ(CH₂)ₜ-A¹, —(C₁-C₁₀)alkyl, —(CH₂)ₜ-A¹, —(CH₂)_{q}—(C₃-C₇)cycloalkyl, —(CH₂)_{q}—Y¹ —(C₁-C₆)alkyl, —(CH₂)_{q}Y¹(CH₂)ₜ-A¹ or —(CH₂)_{q}—Y¹ —(CH₂)t—(C₃-C₇)cycloalkyl;
where the alkyl and cycloalkyl groups in the definition of R¹ are optionally substituted with (C₁-C₄)alkyl, hydroxyl, (C₁-C₄)alkoxy, carboxyl, —CONH₂, —S(O)ₘ(C₁-C₆)alkyl, —CO₂ (C₁-C₄)alkyl ester, 1H-tetrazol-5-yl or 1, 2 or 3 fluoro;
Y¹ is O, S(O)ₘ, —C(O)NX⁶ —, —CH═CH—, —C≡C—, —N(X⁶)C(O)—, —C(O)NX⁶—, —C(O)O—, —OC(O)N(X⁶)— or —OC(O)—;
_{q}is O, 1, 2, 3 or 4;
t is 0, 1, 2 or 3;
said (CH₂)_{q}group and (CH₂)ₜ group may each be optionally substituted with hydroxyl, (C₁-C₄)alkoxy, carboxyl, —CONH₂, —S(O)ₘ(C₁-C₆)alkyl, —CO₂ (C₁-C₄)alkyl ester, 1H-tetrazol-5-yl, 1, 2 or 3 fluoro, or 1 or 2 (C₁-C₄)alkyl;
R² is hydrogen, (C₁-C₈)alkyl, —(C₀-C₃)alkyl-(C₃-C₈)cycloalkyl, —(C₁-C₄)alkyl-A¹ or A¹;
where the alkyl groups and the cycloalkyl groups in the definition of R²are optionally substituted with hydroxyl, —C(O)OX⁶, —C(O)N(X⁶)(X⁶), —N(X⁶)(X⁶), —S(O)ₘ(C₁-C₆)alkyl, —C(O)A¹, —C(O)(X⁶), CF₃, CN or 1, 2 or 3 halogen;
R³ is A¹, (C₁-C₁₀)alkyl, —(C₁-C₆)alkyl-A, —(C₁-C₆)alkyl-(C₃-C₇)cycloalkyl, —(C₁-C₅)alkyl-X—(C₁-C₅)alkyl, —(C₁-C₅)alkyl-X—(C₀-C₅)alkyl-A¹ or —(C₁-C₅)alkyl-X—(C₁-C₅)alkyl-(C₃-C₇)cycloalkyl;
where the alkyl groups in the definition of R³ are optionally substituted with, —S(O)ₘ(C₁-C₆)alkyl, —C(O)OX³, 1, 2, 3, 4 or 5 halogens, or 1, 2 or 3 OX³;
X¹ is O, S(O)ₘ, —N(X²)C(O)—, —C(O)N(X²)—, —OC(O)—, —C(O)O—, —CX²═CX²—, —N(X²)C(O)O—, —OC(O)N(X²)— or —C≡C—;
R⁴ is hydrogen, (C₁-C₆)alkyl or (C₃-C₇)cycloalkyl;
X⁴ is hydrogen or (C₁-C₆)alkyl or X⁴ is taken together with R⁴ and the nitrogen atom to which X⁴ is attached and the carbon atom to which R⁴ is attached and form a five to seven membered ring;
R⁶ is a bond or is 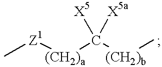
where a and b are independently 0, 1, 2 or 3;
X⁵ and X^{5a} are each independently selected from the group consisting of hydrogen, trifluoromethyl, A¹ and optionally substituted (C₁-C₆)alkyl;
the optionally substituted (C₁-C₆)alkyl in the definition of X⁵ and X^{5a} is optionally substituted with a substituent selected from the group consisting of A¹, OX², —S(O)ₘ(C₁-C₆)alkyl, —C(O)OX², (C₃-C₇)cycloalkyl, —N(X²)(X²) and —C(O)N(X²)(X²);
R⁷ and R⁸are independently hydrogen or optionally substituted (C₁-C₆)alkyl;
where the optionally substituted (C₁-C₆)alkyl in the definition of R⁷ and R⁸ is optionally independently substituted with A¹, —C(O)O—(C₁-C₆)alkyl, —S(O)ₘ(C₁-C₆)alkyl, 1 to 5 halogens, 1 to 3 hydroxy, 1 to 3 —O—C(O)(C₁-C₁₀)alkyl or 1 to 3 (C₁-C₆)alkoxy; or
R⁷ and R⁸ can be taken together to form —(CH₂)ᵣ-L-(CH₂)ᵣ—;
where L is C(X²)(X²), S(O)n, or N(X²);
A¹ in the definition of R¹ is a partially saturated, fully saturated or fully unsaturated 4- to 8-membered ring optionally having 1 to 4 heteroatoms independently selected from the group consisting of oxygen, sulfur and nitrogen, a bicyclic ring system consisting of a partially saturated, fully unsaturated or fully saturated 5- or 6-membered ring, having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
Ain the definition of R², R³, R⁶, R⁷ and R⁸ is independently (C₅-C₇)cycloalkenyl, phenyl or a partially saturated, fully saturated or fully unsaturated 4- to 8-membered ring optionally having 1 to 4 heteroatoms independently selected from the group consisting of oxygen, sulfur and nitrogen, a bicyclic ring system consisting of a partially saturated, fully unsaturated or fully saturated 5- or 6-membered ring, optionally having 1 to 4 heteroatoms independently selected from the group consisting of nitorgen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
A¹ for each occurrence is independently optionally substituted, in one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —C(O)N(X⁶)(X⁶), —C(O)OX⁶, oxo, (C₀-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N(X⁶)(X⁶), —N(X⁶)C(O)(X⁶), —SO₂ N(X⁶)(X⁶), —N(X⁶)SO₂-phenyl, —N(X⁶)SO₂ X⁶, —CONX¹¹X¹², —SO₂ NX ¹ XI², —NX⁶ SO₂ X², —NX⁶ CONX^{11X}¹², —NX⁶ SO₂NX¹¹X¹², —NX⁶C(O)X¹², imidazolyl, thiazolyl or tetrazolyl, provided that if A¹ is optionally substituted with methylenedioxy then it can only be substituted with one methylenedioxy;
where X¹¹ is hydrogen or optionally substituted (C₁-C₆)alkyl;
the optionally substituted (C₁-C₆)alkyl defined for X¹¹ is optionally independently substituted with phenyl, phenoxy, (C₁-C₆)alkoxycarbonyl, —S(O)ₘ(C₁-C₆)alkyl 1 to 5 halogens, 1 to 3 hydroxy, 1 to 3 (C₁ Cl_{0)alkanoyloxy or} 1 to 3 (C₁-C₆)alkoxy;
X¹² is hydrogen, (C₁-C₆)alkyl, phenyl, thiazolyl, imidazolyl, furyl or thienyl, provided that when X¹² is not hydrogen, X¹² is optionally substituted with one to three substituents independently selected from the group consisting of Cl, F, CH₃, OCH₃, OCF₃ and CF₃;
or X¹¹ and X¹²are taken together to form —(CH₂)ᵣ-L¹-(CH₂)ᵣ—;
where L¹ is C(X²)(X²), 0, S(O)ₘ or N(X²);
r for each occurrence is independently 1, 2 or 3;
X² for each occurrence is independently hydrogen, optionally substituted (C₁-C₆)alkyl, or optionally substituted (C₃-C₇)cycloalkyl, where the optionally substituted (C₁-C₆)alkyl and optionally substituted (C₃-C₇)cycloalkyl in the definition of X² are optionally independently substituted with —S(O)ₘ(C₁-C₆)alkyl, —C(O)0X^{3,} 1 to 5 halogens or 1-3 OX³;
X³ for each occurrence is independently hydrogen or (C₁-C₆)alkyl;
X⁶ is independently hydrogen, optionally substituted (C₁-C₈)alkyl, (C₂-C₆)halogenated alkyl, optionally substituted (C₃-C₇)cycloalkyl, (C₃-C₇)-halogenatedcycloalkyl, where optionally substituted (C₁-C₆)alkyl and optionally substituted (C₃-C₇)cycloalkyl in the definition of X⁶ is optionally independently substituted by 1 or 2 (C₁-C₄)alkyl, hydroxyl, (C₁-C₄)alkoxy, carboxyl, CONH₂, —S(O)ₘ(C₁-C₆)alkyl, carboxylate (C₁-C₄)alkyl ester, or 1H-tetrazol-5-yl; or
when there are two X⁶ groups on one atom and both X⁶are independently (C₁-C₆)alkyl, the two (C₁-C₆)alkyl groups may be optionally joined and, together with the atom to which the two X⁶ groups are attached, form a 4- to 9-membered ring optionally having oxygen, sulfur or NX⁷;
X⁷ is hydrogen or (C₁-C₆)alkyl optionally substituted with hydroxyl; and
m for each occurrence is independently 0, 1 or 2;
with the proviso that:
X⁶ and X¹² cannot be hydrogen when it is attached to C(O) or SO₂ in the form C(O)X⁶, C(O)XI², SO₂ X⁶ or SO₂ X¹²; and
when R⁶ is a bond then L is N(X²) and each r in the definition —(CH₂)ᵣ-L-(CH₂)ᵣ— is independently 2 or 3.

36. A method of claim 35 wherein said GHS is 2-amino-N-(1 (R)-benzyloxymethyl-2-(1,3-dioxo-8a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide or a pharmaceutically acceptable salt thereof and said SSRI is sertraline or fluoxetine or a pharmaceutically acceptable salt of sertraline or fluoxetine.

37. A method of claim 36 wherein said GHS is the L-(+)-tartaric acid salt of 2-amino-N-(1(R)-benzyloxymethyl-2-(1,3-dioxo-8a (S)-pyridin-2-ylmethyl-2-(2,2,2-trifluoro-ethyl)-hexahydro-imidazo[1,5-a]pyrazin-7-yl]-2-oxo-ethyl)-2-methyl-propionamide and said SSRI is sertraline hydrochloride.

38. A method of claim 35 wherein said GHS is 2-amino-N-[2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide or a pharmaceutically acceptable salt thereof and said SSRI is sertraline or fluoxetine or a pharmaceutically acceptable salt of sertraline or fluoxetine.

39. A method of claim 38 wherein said GHS is 2-amino-N-[2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide, L-lactate and said SSRI is sertraline hydrochloride.

40. A method of claim 35 wherein said GHS is 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo[4,3-c]pyridin-5-yl)-ethyl)-2-methyl-propionamide or a pharmaceutically acceptable salt thereof and said SSRI is sertraline or fluoxetine or a pharmaceutically acceptable salt of sertraline or fluoxetine.

41. A method of claim 40 wherein said GHS is the L-(+)-tartaric acid salt of 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo[4,3-c]pyridin-5-yl)-ethyl)-2-methyl-propionamide and said SSRI is sertraline hydrochloride.

42. A method for treating musculoskeletal frailty in a mammal comprising administering to said mammal:
a) a pharmaceutical composition of claim 21; or
b) a combination of a growth hormone secretagogue (GHS), prodrug thereof, pharmaceutically acceptable salt of said GHS or of said prodrug or a pharmaceutical composition thereof and an antidepressant, prodrug thereof, pharmaceutically acceptable salt of said antidepressant or said prodrug or a pharmaceutical composition thereof.

43. A method of claim 42 wherein bone healing following facial reconstruction, maxillary reconstruction or mandibular reconstruction is treated, vertebral synostosis is induced or long bone extension is enhanced, the healing rate of a bone graft is enhanced or prosthetic ingrowth is enhanced.

44. A method of claim 42 wherein muscle mass is increased.

45. A kit comprising:
a) a first unit dosage form comprising a GHS, a prodrug thereof or a pharmaceutically acceptable salt of said GHS or said prodrug and a pharmaceutically acceptable carrier, vehicle or diluent;
b) a second unit dosage form comprising an antidepressant, a prodrug thereof or a pharmaceutically acceptable salt of said antidepressant or said prodrug and a pharmaceutically acceptable carrier, vehicle or diluent; and
c) a container.

46. A kit of claim 45 wherein said GHS is 2-amino-N-(1(R)-benzyloxymethyl-2-(1,3-dioxo-8a(S)-pyridin-2-ylmethyl-2-(2,2,2-trifluoro-ethyl)-hexahydro-imidazo[1 ,5-a]pyrazin-7-yl]-2-oxo-ethyl)-2-methyl-propionamide or a pharmaceutically acceptable salt thereof and said antidepressant is an SSRI selected from sertraline or fluoxetine or a pharmaceutically acceptable salt of sertraline or fluoxetine.

47. A kit of claim 46 wherein said GHS is the L-(+)-tartaric acid salt of 2-amino-N-(1(R)-benzyloxymethyl-2-(1,3-dioxo-8a(S)-pyridin-2-ylmethyl-2-(2,2,2-trifluoro-ethyl)-hexahydro-imidazo[1,5-a]pyrazin-7-yl]-2-oxo-ethyl)-2-methyl-propionamide and said SSRI is sertraline hydrochloride.

48. A kit of claim 45 wherein said GHS is 2-amino-N-[2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide or a pharmaceutically acceptable salt thereof and said antidepressant is an SSRI selected from sertraline or fluoxetine or a pharmaceutically acceptable salt of sertraline or fluoxetine.

49. A kit of claim 48 wherein said GHS is the 2-amino-N-[2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl]-isobutyramide, L-tartrate and said SSRI is sertraline hydrochloride.

50. A kit of claim 45 wherein said GHS is 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo[4,3-c]pyridin-5-yl)-ethyl)-2-methyl-propionamide, or a pharmaceutically acceptable salt thereof and said antidepressant is an SSRI selected from sertaline or fluoxetine or a pharmaceutically acceptable salt of sertraline or fluoxetine.

51. A kit of claim 50 wherein said GHS is the L-(+)-tartaric acid salt of 2-amino-N-(1-(R)-(2,4-difluoro-benzyloxymethyl)-2-oxo-2-(3-oxo-3a-(R)-pyridin-2-ylmethyl)-2-(2,2,2-trifluoro-ethyl)-2,3,3a,4,6,7-hexahydro-pyrazolo[4,3-c]pyridin-5-yl)-ethyl)-2-methyl-propionamide and said SSRI is sertraline hydrochloride.

52. A method of treating congestive heart failure in a mammal comprising administering to said mammal:
a) a pharmaceutical composition of claim 21; or
b) a combination of a growth hormone secretagogue (GHS), prodrug thereof, pharmaceutically acceptable salt of said GHS or of said prodrug or a pharmaceutical composition thereof and an antidepressant, prodrug thereof, pharmaceutically acceptable salt of said antidepressant or said prodrug or a pharmaceutical composition thereof.

53. A method of claim 52 wherein said antidepressant is a selective serotonin reuptake inhibitor (SSRI), prodrug thereof or a pharmaceutically acceptable salt of said SSRI or said prodrug.

54. A method of attenuating protein catabolic response after a major operation in a mammal comprising administering to said mammal:
a) a pharmaceutical composition of claim 21; or
b) a combination of a growth hormone secretagogue (GHS), prodrug thereof, pharmaceutically acceptable salt of said GHS or of said prodrug or a pharmaceutical composition thereof and an antidepressant, prodrug thereof, pharmaceutically acceptable salt of said antidepressant or of said prodrug or a pharmaceutical composition thereof.

55. A method of claim 53 wherein said antidepressant is an SSRI, a prodrug thereof or a pharmaceutically acceptable salt of said SSRI or of said prodrug.
